(19) United States
(12) Patent Application Publication
(10) Pub. No.: US 2024/0182597 A1
(43) Pub. Date: Jun. 6, 2024

(54) ANTI-MUC1-C ANTIBODIES AND CAR-T STRUCTURES

(71) Applicant: TeneoBio, Inc., Thousand Oaks, CA (US)

(72) Inventors: Katherine Harris, Thousand Oaks, CA (US); Brian Avanzino, Thousand Oaks, CA (US); Nathan Trinklein, Thousand Oaks, CA (US); Karen Chang, Thousand Oaks, CA (US); Nicole Allen, Thousand Oaks, CA (US)

(21) Appl. No.: 18/547,663

(22) PCT Filed: Feb. 28, 2022
(86) PCT No.: PCT/US2022/018117

§ 371 Date: Aug. 23, 2023

Related U.S. Application Data
(60) Provisional application No. 63/154,618, filed on Feb. 26, 2021.

(51) Int. Cl. C07K 16/30; A61K 39/00; A61P 35/00; C07K 14/725; C12N 5/0783

(52) U.S. Cl. CPC C07K 16/3092; A61K 39/4611; A61K 39/4631; A61K 39/46447; A61P 35/00; C07K 14/7051; C12N 5/0636; C07K 2317/569; C07K 2317/92; C07K 2319/33; C12N 2510/00

(57) ABSTRACT

Anti-MUC1-C antibodies (e.g., UniAbs™) and CAR-T structures are disclosed, along with methods of making such antibodies and CAR-T structures, compositions, including pharmaceutical compositions, comprising such antibodies and CAR-T structures, and their use to treat disorders that are characterized by the expression of MUC1-C.

### CROSS-REFERENCE TO RELATED APPLICATIONS

This application claims priority benefit of the filing date of U.S. Provisional Patent Appliation Ser. No. 63/154,618, filed on Feb. 26, 2021, the disclosure of which is incorporated by reference herein in its entirety.

### FIELD OF THE INVENTION

The present invention concerns antibodies (e.g., UniAbs™) and CAR-T structures that bind to MUC1-C. The invention further concerns methods of making such antibodies and CAR-T structures, compositions, including pharmaceutical compositions, comprising such antibodies and CAR-T structures, and their use to treat disorders that are characterized by the expression of MUC1-C.

### BACKGROUND OF THE INVENTION

### MUC1

Mucin 1 (MUC1) is a heavily glycosylated, single pass type I transmembrane protein. The N-terminal subunit (MUC1-N) and C-terminal subunit (MUC1-C) form a stable heterodimeric complex. MUC1 is highly polymorphic, with greater than 90 isoforms, differing in the number of tandem repeats in the VNTR (variable number tandem repeat) region of the N-terminal subunit. Mucins line the apical surface of epithelial cells in the lungs, stomach, mammary glands, intestines, and several other organs. In healthy tissues, mucins protect the body from infection. Aberrantly glycosylated MUC1 is overexpressed in human epithelial cancers and apical polarity is lost in tumor cells (Sousa et al. 2016, PMC: 4998183, Nath and Mukherjee, 2014, PMID: 5500204). MUC1 can be cleaved by proteases and cleaved MUC1-N is shed from the cell and can trigger inflammation. The non-shed oncogenic MUC1-C subunit is short, containing a 58 amino acid membrane proximal extracellular domain that shows promise as a target for antibody drug conjugates, monoclonal antibodies and CAR-T therapies (Panchamoorthy et al., 2018, PMC: 6124453; Kufe, 2009, PMC: 2951677).

### Heavy Chain Antibodies

In a conventional IgG antibody, the association of the heavy chain and light chain is due in part to a hydrophobic interaction between the light chain constant region and the CH1 constant domain of the heavy chain. There are additional residues in the heavy chain framework 2 (FR2) and framework 4 (FR4) regions that also contribute to this hydrophobic interaction between the heavy and light chains.

It is known, however, that sera of camelids (sub-order Tylopoda which includes camels, dromedaries and llamas) contain a major type of antibodies composed solely of paired H-chains (heavy-chain only antibodies or UniAbs™). The UniAbs™ of Camelidae (Camelus dromedarius, Camelus bactrianus, Lama glama, Lama guanaco, Lama alpaca and Lama vicugna) have a unique structure consisting of a single variable domain (VHH), a hinge region and two constant domains (CH2 and CH3), which are highly homologous to the CH2 and CH3 domains of classical antibodies. These UniAbs™ lack the first domain of the constant region (CH1) which is present in the genome, but is spliced out during mRNA processing. The absence of the CH1 domain explains the absence of the light chain in the UniAbs™, since this domain is the anchoring place for the constant domain of the light chain. Such UniAbs™ naturally evolved to confer antigen-binding specificity and high affinity by three CDRs from conventional antibodies or fragments thereof (Muyldermans, 2001; J Biotechnol 74:277-302; Revets et al., 2005; Expert Opin Biol Ther 5:111-124). Cartilaginous fish, such as sharks, have also evolved a distinctive type of immunoglobulin, designated as IgNAR, which lacks the light polypeptide chains and is composed entirely by heavy chains. IgNAR molecules can be manipulated by molecular engineering to produce the variable domain of a single heavy chain polypeptide (vNARs) (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The ability of heavy chain-only antibodies devoid of light chain to bind antigen was established in the 1960s (Jaton et al. (1968) Biochemistry, 7, 4185-4195). Heavy chain immunoglobulin physically separated from light chain retained 80% of antigen-binding activity relative to the tetrameric antibody. Sitia et al. (1990) Cell, 60, 781-790 demonstrated that removal of the CH1 domain from a rearranged mouse μ gene results in the production of a heavy chain-only antibody, devoid of light chain, in mammalian cell culture. The antibodies produced retained VH binding specificity and effector functions.

Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)).

Mice in which the λ (lambda) light (L) chain locus and/or the λ and κ (kappa) L chain loci have been functionally silenced and antibodies produced by such mice are described in U.S. Pat. Nos. 7,541,513 and 8,367,888. Recombinant production of heavy chain-only antibodies in mice and rats has been reported, for example, in WO2006008548; U.S. Application Publication No. 20100122358; Nguyen et al., 2003, Immunology; 109(1), 93-101; Briiggemann et al., Crit. Rev. Immunol.; 2006, 26(5):377-90; and Zou et al., 2007, J Exp Med; 204(13): 3271-3283. The production of knockout rats via embryo microinjections of zinc-finger nucleases is described in Geurts et al., 2009, Science, 325(5939):433. Soluble heavy chain-only antibodies and transgenic rodents comprising a heterologous heavy chain locus producing such antibodies are described in U.S. Pat. Nos. 8,883,150 and 9,365,655. CAR-T structures comprising single-domain antibodies as binding (targeting) domains are described, for example, in Iri-Sofia et al., 2011, Experimental Cell Research 317:2630-2641 and Jamnani et al., 2014, Biochim Biophys Acta, 1840:378-386.

### SUMMARY OF THE INVENTION

Aspects of the invention include antibodies that bind to MUC1-C, comprising a heavy chain variable region comprising: (a) a CDR1 sequence comprising two or fewer substitutions in any one of the amino acid sequences of SEQ ID NOs: 1 or 4; and/or (b) a CDR2 sequence comprising two or fewer substitutions in any one of the amino acid sequences of SEQ ID NOs: 2 or 5; and/or (c) a CDR3 sequence comprising two or fewer substitutions in any one of the amino acid sequences of SEQ ID NOs: 3 or 6. In some embodiments, the CDR1, CDR2, and CDR3 sequences are present in a human framework. In some embodiments, an antibody further comprises a heavy chain constant region sequence in the absence of a CH1 sequence.

In some embodiments, an antibody comprises: (a) a CDR1 sequence selected from the group consisting of SEQ ID NOs: 1 and 4; and/or (b) a CDR2 sequence selected from the group consisting of SEQ ID NOs: 2 and 5; and/or (c) a CDR3 sequence selected from the group consisting of SEQ ID NOs: 3 and 6.

In some embodiments, an antibody comprises: (a) a CDR1 sequence selected from the group consisting of SEQ ID NOs: 1 and 4; and (b) a CDR2 sequence selected from the group consisting of SEQ ID NOs: 2 and 5; and (c) a CDR3 sequence selected from the group consisting of SEQ ID NOs: 3 and 6.

In some embodiments, an antibody comprises: (a) a CDR1 sequence of SEQ ID NO: 1, a CDR2 sequence of SEQ ID NO: 2, and a CDR3 sequence of SEQ ID NO: 3; or (b) a CDR1 sequence of SEQ ID NO: 4, a CDR2 sequence of SEQ ID NO: 5, and a CDR3 sequence of SEQ ID NO: 6.

In some embodiments, an antibody comprises a heavy chain variable region having at least 95% sequence identity to any of the sequences of SEQ ID NOs: 7-8. In some embodiments, an antibody comprises a heavy chain variable region sequence selected from the group consisting of SEQ ID NOs: 7-8. In some embodiments, an antibody comprises a heavy chain variable region sequence of SEQ ID NO: 7. In some embodiments, an antibody comprises a heavy chain variable region sequence of SEQ ID NO: 8.

Aspects of the invention include antibodies that bind to MUC1-C, comprising a heavy chain variable region comprising CDR1, CDR2 and CDR3 sequences in a human VH framework, wherein the CDR sequences are sequences having two or fewer substitutions in a CDR sequence selected from the group consisting of SEQ ID NOs: 1-6.

In some embodiments, an antibody comprises a heavy chain variable region comprising CDR1, CDR2 and CDR3 sequences in a human VH framework, wherein the CDR sequences are selected from the group consisting of SEQ ID NOs: 1-6.

Aspects of the invention include antibodies that bind to MUC1-C, comprising a heavy chain variable region comprising: (a) a CDR1 sequence of SEQ ID NO: 1, a CDR2 sequence of SEQ ID NO: 2, and a CDR3 sequence of SEQ ID NO: 3, in a human VH framework; or (b) a CDR1 sequence of SEQ ID NO: 4, a CDR2 sequence of SEQ ID NO: 5, and a CDR3 sequence of SEQ ID NO: 6, in a human VH framework.

In some embodiments, an antibody is in a CAR-T format. In some embodiments, an antibody is multi-specific. In some embodiments, an antibody is bispecific. In some embodiments, an antibody binds to two different MUC1-C proteins. In some embodiments, an antibody binds to two different epitopes on the same MUC1-C protein. In some embodiments, an antibody binds to an effector cell. In some embodiments, an antibody binds to a T-cell antigen. In some embodiments, an antibody binds to CD3.

In some embodiments, an antibody comprises: (a) a heavy chain variable region comprising: (i) a CDR1 sequence of SEQ ID NO: 9, a CDR2 sequence of SEQ ID NO: 10, and a CDR3 sequence of SEQ ID NO: 11, in a human VH framework; or (ii) a CDR1 sequence of SEQ ID NO: 12, a CDR2 sequence of SEQ ID NO: 13, and a CDR3 sequence of SEQ ID NO: 14, in a human VH framework; and (b) a light chain variable region comprising a CDR1 sequence of SEQ ID NO: 15, a CDR2 sequence of SEQ ID NO: 16, and a CDR3 sequence of SEQ ID NO: 17, in a human VL framework.

In some embodiments, an antibody comprises: (a) a heavy chain variable region comprising: (i) a heavy chain variable region sequence having at least 95% sequence identity to SEQ ID NO: 18; or (ii) a heavy chain variable region sequence having at least 95% sequence identity to SEQ ID NO: 19; and (b) a light chain variable region sequence having at least 95% sequence identity to SEQ ID NO: 20.

In some embodiments, an antibody comprises: (a) a heavy chain variable region comprising: (i) a heavy chain variable region sequence comprising SEQ ID NO: 18; or (ii) a heavy chain variable region sequence comprising SEQ ID NO: 19; and (b) a light chain variable region sequence comprising SEQ ID NO: 20.

Aspects of the invention include bispecific three-chain antibody-like molecules (TCAs) that binds to MUC1-C and CD3, comprising: (a) a first polypeptide consisting of SEQ ID NO: 32; (b) a second polypeptide selected from the group consisting of: SEQ ID NO: 33 and SEQ ID NO: 42; and (c) a third polypeptide selected from the group consisting of: SEQ ID NO: 34, SEQ ID NO: 35, SEQ ID NO: 36, SEQ ID NO: 37, SEQ ID NO: 38, and SEQ ID NO: 39.

Aspects of the invention include CAR-T cells comprising a CAR comprising an extracellular antigen-binding domain that binds to MUC1-C, comprising a heavy chain variable region comprising: (a) a CDR1 sequence of SEQ ID NO: 1, a CDR2 sequence of SEQ ID NO: 2, and a CDR3 sequence of SEQ ID NO: 3; or (b) a CDR1 sequence of SEQ ID NO: 4, a CDR2 sequence of SEQ ID NO: 5, and a CDR3 sequence of SEQ ID NO: 6. In some embodiments, the extracellular antigen-binding domain that binds to MUC1-C comprises a heavy chain variable region having at least 95% sequence identity to any of the sequences of SEQ ID NOs: 7-8. In some embodiments, the extracellular antigen-binding domain that binds to MUC1-C comprises a heavy chain variable region sequence selected from the group consisting of SEQ ID NOs: 7-8. In some embodiments, the extracellular antigen-binding domain that binds to MUC1-C comprises a heavy chain variable region sequence of SEQ ID NO: 7. In some embodiments, the extracellular antigen-binding domain that binds to MUC1-C comprises a heavy chain variable region sequence of SEQ ID NO: 8.

Aspects of the invention include pharmaceutical compositions comprising an antibody as described herein, or a CAR-T cell as described herein.

Aspects of the invention include methods for the treatment of a disorder characterized by expression of MUC1-C, comprising administering to a subject with said disorder an antibody as described herein, a CAR-T cell as described herein, or a pharmaceutical composition as described herein. In some embodiments, the disorder is a cancer. In some embodiments, the cancer is a carcinoma. In some embodiments, the carcinoma is an adenocarcinoma or a squamous cell carcinoma. In some embodiments, the carcinoma is selected from the group consisting of: breast, non-small cell lung (NSCL), small cell lung (SSC), mesothelioma, renal cell, colorectal, ovarian, head and neck squamous cell, nasopharyngeal, gastric, prostatic, pancreatic, esophageal, and cervical carcinoma. In some embodiments, the cancer is a hematological cancer. In some embodiments, the hematological cancer is a myeloma. In some embodiments, the myeloma is multiple myeloma (MM). In some embodiments, the hematological cancer is a leukemia. In some embodiments, the leukemia is chronic myeloid leukemia (CML). In some embodiments, the hematological cancer is a lymphoma.

Aspects of the invention include polynucleotides encoding an antibody as described herein, or a CAR of a CAR-T cell as described herein.

Aspects of the invention include vectors comprising the polynucleotide as described herein.

Aspects of the invention include cells comprising the vectors as described herein.

Aspects of the invention include methods of producing an antibody as described herein, the methods comprising growing a cell as described herein under conditions permissive for expression of the antibody, and isolating the antibody from the cell and/or a cell culture medium in which the cell is grown.

Aspects of the invention include methods of making an antibody as described herein, the methods comprising immunizing a UniRat animal with MUC1-C and identifying MUC1-C-binding heavy chain sequences.

Aspects of the invention include methods of treatment, comprising administering to an individual in need an effective dose of an antibody as described herein, a CAR-T cell as described herein, or a pharmaceutical composition as described herein.

Aspects of the invention include use of an antibody as described herein or a CAR-T cell as described herein in the preparation of a medicament for the treatment of a disease or disorder in an individual in need.

Aspects of the invention include kits for treating a disease or disorder in an individual in need, comprising an antibody as described herein, a CAR-T cell as described herein, or a pharmaceutical composition as described herein, and instructions for use. In some embodiments, a kit comprises at least one additional reagent. In some embodiments, the at least one additional reagent comprises a chemotherapeutic drug.

These and further aspects will be further explained in the rest of the disclosure, including the Examples.

### BRIEF DESCRIPTION OF THE DRAWINGS

FIG. 1 is a table summarizing MUC1-C binding data for the indicated antibody constructs to MUC1-C+ Raji cells and negative control cells.

FIG. 2 is a graph showing cell binding as a function of antibody concentration for the indicated antibody constructs, in MUC1-C+ Raji cells.

FIG. 3 is a table summarizing cell binding EC50 values of the indicated antibody constructs on MUC1-C+ Raji cells.

FIG. 4, Panel A, is a schematic diagram of a CAR-T structure comprising an anti-MUC1-C extracellular binding domain.

FIG. 4, Panel B, is a graph showing T-cell activity of Jurkat cells transfected with an anti-MUC1-C CAR construct in accordance with one embodiment of the invention.

FIG. 4, Panel C, is a graph showing T-cell activity of Jurkat cells transfected with an anti-MUC1-C CAR construct in accordance with one embodiment of the invention.

FIG. 5, Panel A, is a graph showing tumor progression as a function of days post CAR-T infusion for the indicated antibody constructs.

FIG. 5, Panel B, is a bar chart showing the area under curve of the graph shown in FIG. 5, Panel A, for the indicated antibody constructs.

FIG. 6, Panel A, is a graph showing in vivo T-cell count in blood as a function of days post CAR-T infusion for the indicated antibody constructs.

FIG. 6, Panel B, is a bar chart showing the area under curve of the graph shown in FIG. 6, Panel A, for the indicated antibody constructs.

FIG. 7, Panel A, is a graph showing tumor progression as a function of days post CAR-T infusion for the indicated antibody constructs.

FIG. 7, Panel B, is a line graph showing T-cell persistence in blood measured over time for the indicated antibody constructs.

FIG. 8, Panel A, is a graph showing in vivo T-cell count in blood as a function of days post CAR-T infusion for the indicated antibody constructs.

FIG. 8, Panel B, is a bar chart showing the area under curve of the graph shown in FIG. 6, Panel A, for the indicated antibody constructs.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

Antibodies, also referred to as immunoglobulins, conventionally comprise at least one heavy chain and one light chain, where the amino terminal domain of the heavy and light chains is variable in sequence, hence is commonly referred to as a variable region domain, or a variable heavy (VH) or variable light (VL) domain. The two domains conventionally associate to form a specific binding region, although as will be discussed here, specific binding can also be obtained with heavy chain-only variable sequences, and a variety of non-natural configurations of antibodies are known and used in the art.

A “functional” or “biologically active” antibody or antigen-binding molecule (including heavy chain-only antibodies and multi-specific (e.g., bispecific) three-chain antibody-like molecules (TCAs, described herein) is one capable of exerting one or more of its natural activities in structural, regulatory, biochemical or biophysical events. For example, a functional antibody or other binding molecule, e.g., a TCA, may have the ability to specifically bind an antigen and the binding may in turn elicit or alter a cellular or molecular event such as signal transduction or enzymatic activity. A functional antibody or other binding molecule, e.g., a TCA, may also block ligand activation of a receptor or act as an agonist or antagonist. The capability of an antibody or other binding molecule, e.g., a TCA, to exert one or more of its natural activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), heavy chain-only antibodies, three chain antibodies, TCAs, single chain FAT (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule, or an immunologically active portion of any of these polypeptides, i.e., a polypeptide that comprises an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgAl and IgA2) or subclass of immunoglobulin molecule, including engineered subclasses with altered Fc portions that provide for reduced or enhanced effector cell activity. Light chains of the subject antibodies can be kappa light chains (Vkappa) or lambda light chains (Vlambda). The immunoglobulins can be derived from any species. In one aspect, the immunoglobulin is of largely human origin.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an

antibody which are responsible for antigen-binding. The hypervariable region generally comprises amino acid residues from a “complementarity-determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). In some embodiments, “CDR” means a complementarity-determining region of an antibody as defined in Lefranc, M P et al., IMGT, the International ImMunoGeneTics database, Nucleic Acids Res., 27:209-212 (1999). “Framework Region” or “FR” residues are those variable domain residues other than the hypervariable region/CDR residues as herein defined.

Exemplary CDR designations are shown herein; however, one of skill in the art will understand that a number of definitions of the CDRs are commonly in use, including the Kabat definition (see “Zhao et al. A germline knowledge based computational approach for determining antibody complementarity determining regions.” Mol Immunol. 2010;47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001;309:657-670; Ofran et al. “Automated identification of complementarity determining regions (CDRs) reveals peculiar characteristics of CDRs and B-cell epitopes.” J Immunol. 2008;181:6230-6235; Almagro “Identification of differences in the specificity-determining residues of antibodies that recognize antigens of different size: implications for the rational design of antibody repertoires.” J Mol Recognit. 2004;17:132-143; and Padlanet al. “Identification of specificity-determining residues in antibodies.” Faseb J. 1995;9:133-139., each of which is herein specifically incorporated by reference.

The terms “heavy chain-only antibody,” and “heavy chain antibody” are used interchangeably herein and refer, in the broadest sense, to antibodies, or one or more portions of an antibody, e.g., one or more arms of an antibody, lacking the light chain of a conventional antibody. The terms specifically include, without limitation, homodimeric antibodies comprising the VH antigen-binding domain and the CH2 and CH3 constant domains, in the absence of the CH1 domain; functional (antigen-binding) variants of such antibodies, soluble VH variants, Ig-NAR comprising a homodimer of one variable domain (V-NAR) and five C-like constant domains (C-NAR) and functional fragments thereof; and soluble single domain antibodies (sUniDabs™). In one embodiment, a heavy chain-only antibody is composed of a variable region antigen-binding domain composed of framework 1, CDR1, framework 2, CDR2, framework 3, CDR3, and framework 4. In another embodiment, a heavy chain-only antibody is composed of an antigen-binding domain, at least part of a hinge region and CH2 and CH3 domains. In another embodiment, a heavy chain-only antibody is composed of an antigen-binding domain, at least part of a hinge region and a CH2 domain. In a further embodiment, a heavy chain-only antibody is composed of an antigen-binding domain, at least part of a hinge region and a CH3 domain. Heavy chain-only antibodies in which the CH2 and/or CH3 domain is truncated are also included herein. In a further embodiment, a heavy chain is composed of an antigen binding domain, and at least one CH (CH1, CH2, CH3, or CH4) domain but no hinge region. A heavy chain-only antibody can be in the form of a dimer, in which two heavy chains are disulfide bonded or otherwise, covalently or non-covalently, attached with each other. The heavy chain-only antibody may belong to the IgG subclass, but antibodies belonging to other subclasses, such as IgM, IgA, IgD and IgE subclass, are also included herein. In a particular embodiment, a heavy chain antibody is of the IgG1, IgG2, IgG3, or IgG4 subtype, in particular the IgG1 or IgG4 subtype. In one embodiment, a heavy-chain antibody is of the IgG4 subtype, wherein one or more of the CH domains is modified to alter an effector function of the antibody. In one embodiment, the heavy-chain antibody is of the IgG1 or IgG4 subtype, wherein one or more of the CH domains is modified to alter an effector function of the antibody. Modifications of CH domains that alter effector function are further described herein. Non-limiting examples of heavy-chain antibodies are described, for example, in WO2018/039180, the disclosure of which is incorporated herein by reference in its entirety.

In some embodiments, the heavy chain-only antibodies herein are used as a binding (targeting) domain of a chimeric antigen receptor (CAR). The definition specifically includes human heavy chain-only antibodies produced by human immunoglobulin transgenic rats (UniRat™), called UniAbs™. The variable regions (VH) of UniAbs™ are called UniDabs™, and are versatile building blocks that can be linked to Fc regions or serum albumin for the development of novel therapeutics with multi-specificity, increased potency and extended half-life. Since the homodimeric UniAbs™ lack a light chain and thus a VL domain, the antigen is recognized by one single domain, i.e., the variable domain of the heavy chain of a heavy-chain antibody (VH or VHH).

An “intact antibody chain” as used herein is one comprising a full length variable region and a full length constant region (Fc). An intact “conventional” antibody comprises an intact light chain and an intact heavy chain, as well as a light chain constant domain (CL) and heavy chain constant domains, CH1 hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as α, δ, ϵ, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called κ (kappa) and λ (lambda), based on the amino acid sequences of their constant domains. Antibodies in accordance with embodiments of the invention can comprise kappa light chain sequences or lambda light chain sequences.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIHB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1 q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour KL. et al., 1999 Eur J Immunol. 29(8):2613-24; and Shields R L. et al., 2001. J Biol Chem. 276(9):6591-604). The human IgG4 Fc amino acid sequence (UniProtKB No. P01861) is provided herein as SEQ ID NO: 22. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a Clq-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

In some embodiments, an antibody comprises a variant human IgG4 CH3 domain sequence comprising a T366W mutation, which can optionally be referred to herein as an IgG4 CH3 knob sequence. In some embodiments, an antibody comprises a variant human IgG4 CH3 domain sequence comprising a T366S mutation, an L368A mutation, and a Y407V mutation, which can optionally be referred to herein as an IgG4 CH3 hole sequence. The IgG4 CH3 mutations described herein can be utilized in any suitable manner so as to place a “knob” on a first heavy chain constant region of a first monomer in an antibody dimer, and a “hole” on a second heavy chain constant region of a second monomer in an antibody dimer, thereby facilitating proper pairing (heterodimerization) of the desired pair of heavy chain polypeptide subunits in the antibody.

In some embodiments, an antibody comprises a heavy chain polypeptide subunit comprising a variant human IgG4 Fc region comprising an S228P mutation, an F234A mutation, an L235A mutation, and a T366W mutation (knob). In some embodiments, and antibody comprises a heavy chain polypeptide subunit comprising a variant human IgG4 Fc region comprising an S228P mutation, an F234A mutation, an L235A mutation, a T366S mutation, an L368A mutation, and a Y407V mutation (hole).

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, an antibody having an Fc region according to this invention can comprise an antibody with or without K447.

Aspects of the invention include antibodies comprising a heavy chain-only variable region in a monovalent or bivalent configuration. As used herein, the term “monovalent configuration” as used in reference to a heavy chain-only variable region domain means that only one heavy chain-only variable region domain is present, having a single binding site. In contrast, the term “bivalent configuration” as used in reference to a heavy chain-only variable region domain means that two heavy chain-only variable region domains are present (each having a single binding site), and are connected by a linker sequence. Non-limiting examples of linker sequences are discussed further herein, and include, without limitation, GS linker sequences of various lengths. When a heavy chain-only variable region is in a bivalent configuration, each of the two heavy chain-only variable region domains can bind to the same antigen, or to different antigens (e.g., to different epitopes on the same protein; to two different proteins, etc.). However, unless specifically noted otherwise, a heavy chain-only variable region denoted as being in a “bivalent configuration” is understood to contain two identical heavy chain-only variable region domains, connected by a linker sequence, wherein each of the two identical heavy chain-only variable region domains binds to the same target antigen.

Aspects of the invention include antibodies having multi-specific configurations, which include, without limitation, bispecific, trispecific, etc. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 40) (n=1) and GGGGSGGGGS (SEQ ID NO: 41) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety.

The term “three-chain antibody-like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (e.g., two antigen binding domains) that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

A TCA binding compound makes use of a “heavy chain only antibody” or “heavy chain antibody” or “heavy chain polypeptide” which, as used herein, mean a single chain antibody comprising heavy chain constant regions CH2 and/or CH3 and/or CH4 but no CH1 domain. In one embodiment, the heavy chain antibody is composed of an antigen-binding domain, at least part of a hinge region and CH2 and CH3 domains. In another embodiment, the heavy chain antibody is composed of an antigen-binding domain, at least part of a hinge region and a CH2 domain. In a further embodiment, the heavy chain antibody is composed of an antigen-binding domain, at least part of a hinge region and a CH3 domain. Heavy chain antibodies in which the CH2 and/or CH3 domain is truncated are also included herein. In a further embodiment, the heavy chain is composed of an antigen binding domain, and at least one CH (CH1, CH2, CH3, or CH4) domain but no hinge region. The heavy chain only antibody can be in the form of a dimer, in which two heavy chains are disulfide bonded or otherwise covalently or non-covalently attached to each other, and can optionally include an asymmetric interface (e.g., a knobs-in-holes (KiH) interface) between one or more of the CH domains to facilitate proper pairing between polypeptide chains. The heavy-chain antibody may belong to the IgG subclass, but antibodies belonging to other subclasses, such as IgM, IgA, IgD and IgE subclass, are also included herein. In a particular embodiment, the heavy chain antibody is of the IgG1, IgG2, IgG3, or IgG4 subtype, in particular the IgG1 subtype or the IgG4 subtype. Non-limiting examples of a TCA binding compound are described in, for example, WO2017/223111 and WO2018/052503, the disclosures of which are incorporated herein by reference in their entirety.

Heavy-chain antibodies constitute about one fourth of the IgG antibodies produced by the camelids, e.g., camels and llamas (Hamers-Casterman C., et al. Nature. 363, 446-448 (1993)). These antibodies are formed by two heavy chains but are devoid of light chains. As a consequence, the variable antigen binding part is referred to as the VHH domain and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The term “MUC1” as used herein refers to a membrane-bound protein that is a member of the mucin family. Mucins are O-glycosylated proteins that play an essential role in forming protective mucous barriers on epithelial surfaces, and also play a role in intracellular signaling. MUC1 is expressed on the apical surface of epithelial cells that line the mucosal surfaces of many different tissues.

The term “MUC1-N” refers to the N-terminal, or alpha subunit, of MUC1, and the term “MUC1-C” refers to the C-terminal, or beta subunit, or MUC1. In the case of human MUC1 (UniProt P15941), MUC1-N includes amino acid residues 24-1,097 of MUC1, and MUC1-C includes amino acid residues 1,098-1,255 of MUC1 (UniProt P15941).

The terms “MUC1”, “MUC1-N” and “MUC1-C” include a MUC1, MUC1-N or MUC1-C protein of any human and non-human animal species, and specifically include human MUC1, MUC1-N and MUC1-C, as well as MUC1, MUC1-N and MUC1-C of non-human mammals.

The term “human MUC1” as used herein includes any variants, isoforms and species homologs of human MUC1 (UniProt P15941), regardless of its source or mode of preparation. Thus, “human MUC1” includes human MUC1 naturally expressed by cells and MUC1 expressed on cells transfected with the human MUC1 gene.

The terms “anti-MUC1-C heavy chain-only antibody,” “MUC1-C heavy chain-only antibody,” “anti-MUC1-C heavy chain antibody” and “MUC1-C heavy chain antibody” are used herein interchangeably to refer to a heavy chain-only antibody as hereinabove defined, immunospecifically binding to MUC1-C, including human MUC1-C, as hereinabove defined. The definition includes, without limitation, human heavy chain antibodies produced by transgenic animals, such as transgenic rats or transgenic mice expressing human immunoglobulin, including UniRats™ producing human anti-MUC1-C UniAb™ antibodies, as hereinabove defined.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies of the invention include multi-specific antibodies. Multi-specific antibodies have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. The terms “multi-specific antibody,” “multi-specific heavy chain-only antibody,” “multi-specific heavy chain antibody,” and “multi-specific UniAb™” are used herein in the broadest sense and cover all antibodies with more than one binding specificity. The multi-specific heavy chain anti-MUC1-C antibodies of the present invention specifically include antibodies immunospecifically binding to two or more non-overlapping epitopes on a MUC1-C protein, such as a human MUC1-C (i.e., bivalent and biparatopic). The multi-specific heavy chain anti-MUC1-C antibodies of the present invention also specifically include antibodies immunospecifically binding to an epitope on a MUC1-C protein, such as human MUC1-C, and to an epitope on a different protein, such as, for example, a CD3 protein, such as human CD3 (i.e., bivalent and biparatopic). The multi-specific heavy chain anti-MUC1-C antibodies of the present invention also specifically include antibodies immunospecifically binding to two or more non-overlapping or partially overlapping epitopes on a MUC1-C protein, such as a human MUC1-C protein, and to an epitope on a different protein, such as, for example, a CD3 protein, such as human CD3 protein (i.e., trivalent and biparatopic).

Antibodies of the invention include monospecific antibodies, having one binding specificity. Monospecific antibodies specifically include antibodies comprising a single binding specificity, as well as antibodies comprising more than one binding unit having the same binding specificity. The terms “monospecific antibody,” “monospecific heavy chain-only antibody,” “monospecific heavy chain antibody,” and “monospecific UniAb™” are used herein in the broadest sense and cover all antibodies with one binding specificity. The monospecific heavy chain anti-MUC1-C antibodies of the present invention specifically include antibodies immunospecifically binding to one epitope on a MUC1-C protein, such as a human MUC1-C protein (monovalent and monospecific). The monospecific heavy chain anti-MUC1-C antibodies of the present invention also specifically include antibodies having more than one binding unit (e.g., multivalent antibodies) immunospecifically binding to an epitope on a MUC1-C protein, such as human MUC1-C. For example, a monospecific antibody in accordance with embodiments of the invention can include a heavy chain variable region comprising two antigen-binding domains, wherein each antigen-binding domain binds to the same epitope on a MUC1-C protein (i.e., bivalent and monospecific).

An “epitope” is the site on the surface of an antigen molecule to which a single antibody molecule binds. Generally, an antigen has several or many different epitopes and reacts with many different antibodies. The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

“Polyepitopic specificity” refers to the ability to specifically bind to two or more different epitopes on the same or different target(s). As noted above, the present invention specifically includes anti-MUC1-C heavy chain antibodies with polyepitopic specificities, i.e., anti-MUC1-C heavy chain antibodies binding to one or more non-overlapping epitopes on a MUC1-C protein, such as a human MUC1-C; and anti-MUC1-C heavy chain antibodies binding to one or more epitopes on a MUC1-C protein and to an epitope on a different protein, such as, for example, a CD3 protein. The term “non-overlapping epitope(s)” or “non-competitive epitope(s)” of an antigen is defined herein to mean epitope(s) that are recognized by one member of a pair of antigen-specific antibodies but not the other member. Pairs of antibodies, or antigen-binding regions targeting the same antigen on a multi-specific antibody, recognizing non-overlapping epitopes, do not compete for binding to that antigen and are able to bind that antigen simultaneously.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in all number of different formats, using either labeled antigen or labeled antibody. Usually, the antigen is immobilized on a 96-well plate, and the ability of unlabeled antibodies to block the binding of labeled antibodies is measured using radioactive or enzyme labels.

The term “valent” as used herein refers to a specified number of binding sites in an antibody molecule.

A “monovalent” antibody has one binding site. Thus, a monovalent antibody is also monospecific.

A “multi-valent” antibody has two or more binding sites. Thus, the terms “bivalent”, “trivalent”, and “tetravalent” refer to the presence of two binding sites, three binding sites, and four binding sites, respectively. Thus, a bispecific antibody according to the invention is at least bivalent and may be trivalent, tetravalent, or otherwise multi-valent. A bivalent antibody in accordance with embodiments of the invention may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB), tri-specific antibodies, and the like.

The term “three-chain antibody like molecule” or “TCA” is used herein to refer to antibody-like molecules comprising, consisting essentially of, or consisting of three polypeptide subunits, two of which comprise, consist essentially of, or consist of one heavy chain and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy chain-only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments. A TCA protein makes use of a heavy chain-only antibody as hereinabove defined.

The term “chimeric antigen receptor” or “CAR” is used herein in the broadest sense to refer to an engineered receptor, which grafts a desired binding specificity (e.g., the antigen-binding region of a monoclonal antibody or other ligand) to membrane-spanning and intracellular-signaling domains. Typically, the receptor is used to graft the specificity of a monoclonal antibody onto a T-cell to create a chimeric antigen receptors (CAR). (J Natl Cancer Inst, 2015; 108(7):dvj439; and Jackson et al., Nature Reviews Clinical Oncology, 2016; 13:370-383). CAR-T cells are T-cells that have been genetically engineered to produce an artificial T-cell receptor for use in immunotherapy. In one embodiment, “CAR-T cell” means a therapeutic T-cell expressing a transgene encoding one or more chimeric antigen receptors comprised minimally of an extracellular domain, a transmembrane domain, and at least one cytosolic domain.

The term “human antibody” is used herein to include antibodies having variable and constant regions derived from human germline immunoglobulin sequences. The human antibodies herein may include amino acid residues not encoded by human germline immunoglobulin sequences, e.g., mutations introduced by random or site-specific mutagenesis in vitro or by somatic mutation in vivo. The term “human antibody” specifically includes heavy chain-only antibodies having human heavy chain variable region sequences, produced by transgenic animals, such as transgenic rats or mice, in particular UniAbs™ produced by UniRats™, as defined above.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the invention that have been engineered to produce such chimeric antibodies.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T-cell receptors or FcRs and perform effector functions. Preferably, the cells express at least FcγRIII and perform ADCC effector function. Examples of human leukocytes which mediate ADCC include natural killer (NK) cells, monocytes, cytotoxic T-cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1 q binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, Calif.) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12(8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The term “characterized by expression of MUC1-C” broadly refers to any disease or disorder in which MUC1-C expression is associated with or involved with one or more pathological processes that are characteristic of the disease or disorder. Such disorders include, but are not limited to, solid tumors and hematological malignancies, such as those described further herein. In some embodiments, a disease or disorder characterized by expression of MUC1-C includes cancers of epithelial origin, i.e., carcinomas, including adenocarcinomas and squamous cell carcinomas. Non-limiting examples of carcinomas include: breast, non-small cell lung (NSCL), small cell lung (SSC), mesothelioma, renal cell, colorectal, ovarian, head and neck squamous cell, nasopharyngeal, gastric, prostatic, pancreatic, esophageal, and cervical carcinomas. In some embodiments, a disease or disorder characterized by expression of MUC1-C includes hematological malignancies, i.e., myelomas, leukemias, and lymphomas. Non-limiting examples of hematological malignancies include multiple myeloma and chronic myeloid leukemia (CML).

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

### II. Detailed Description

### Anti-MUC1-C Antibodies

The present invention provides a family of closely related antibodies that bind to human MUC1-C. The antibodies of this family comprise a set of CDR sequences as defined herein and shown in Table 1, and are exemplified by the provided heavy chain CDR1, CDR2 and CDR3 sequences set forth in Table 2, and the heavy chain variable region (VH) sequences of SEQ ID NOs: 7 and 8 set forth in Table 3. The family of antibodies provides a number of benefits that contribute to utility as clinically therapeutic agent(s). The antibodies include members with a range of binding affinities, allowing the selection of a specific sequence with a desired binding affinity.

TABLE 1
Anti-MUC1-C heavy chain antibody
unique CDR amino acid sequences.

| SEQ_aa_CDR1 | SEQ_aa_CDR2 | SEQ_aa_CDR3 |
|---|---|---|
| GFAFSGNS | ITSSGRSI | ATGGTGTSLFDY |
| (SEQ ID NO: 1) | (SEQ ID NO: 2) | (SEQ ID NO: 3) |
| GFTFSSHS | ISSSSNIK | ATGGTGITVLDY |
| (SEQ ID NO: 4) | (SEQ ID NO: 5) | (SEQ ID NO: 6) |

TABLE 2
Anti-MUC1-C heavy chain antibody CDR1,
CDR2 and CDR3 amino acid sequences.

| Clone | | | |
|---|---|---|---|
| ID # | SEQ_aa_CDR1 | SEQ_aa_CDR2 | SEQ_aa_CDR3 |
| 375747 | GFAFSGNS | ITSSGRSI | ATGGTGTSLFDY |
| | (SEQ ID NO: 1) | (SEQ ID NO: 2) | (SEQ ID NO: 3) |
| 375505 | GFTFSSHS | ISSSSNIK | ATGGTGITVLDY |
| | (SEQ ID NO: 4) | (SEQ ID NO: 5) | (SEQ ID NO: 6) |

TABLE 3
Anti-MUC1-C heavy chain antibody
variable domain amino acid sequences.

| Clone | | SEQ ID |
|---|---|---|
| ID # | SEQ_aa_FR1_FR4 | NO. |
| 375747 | EVQLVESGGGLVQPGGSLRLSCTASGFAF | 7 |
| | SGNSMNWVRQAPGKGLEWVAFITSSGRSI | |
| | KYADSVKGRFTISRDNAKNSLYLQMNTLR | |
| | DEDTALYYCATGGTGTSLFDYRGQGTLVT | |
| | VSS | |
| 375505 | EVQLVESGGGLVQPGGSLRLSCAASGFTF | 8 |
| | SSHSMNWVRQAPGKGLEWVSFISSSSNIK | |
| | KYADSVKGRFTISRDNAKNSLFLQMNSLR | |
| | DEDTAVYYCATGGTGITVLDYRGQGTLVT | |
| | VSS | |

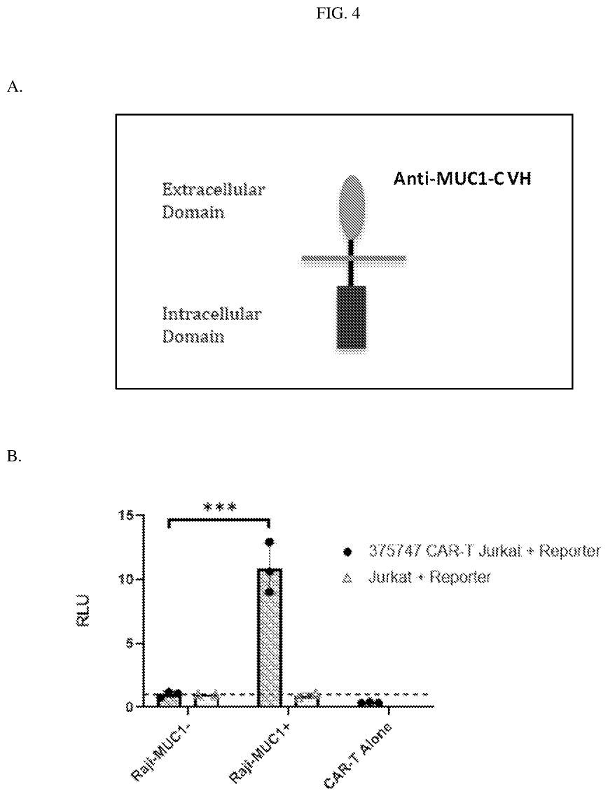

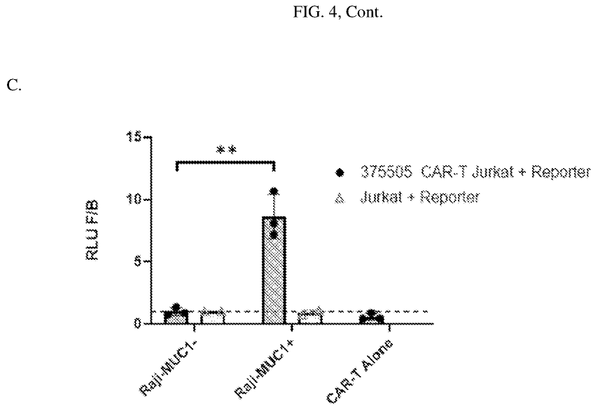

A suitable antibody may be selected from those provided herein for development and therapeutic or other use, including, without limitation, use as a bispecific antibody, or part of a CAR-T structure, as shown, for example, in FIG. 4.

Determination of affinity for a candidate protein can be performed using methods known in the art, such as Biacore measurements. Members of the antibody family may have an affinity for MUC1-C with a Kd of from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating, e.g., blocking, a MUC1-C biological activity, including in vitro assays, pre-clinical models, and clinical trials, as well as assessment of potential toxicity.

Members of the antibody family herein are not cross-reactive with the MUC1-C protein of Cynomolgus macaque, but can be engineered to provide cross-reactivity with the MUC1-C protein of Cynomolgus macaque, or with the MUC1-C of any other animal species, if desired.

The family of MUC1-C-specific antibodies herein comprises a VH domain, comprising CDR1, CDR2 and CDR3 sequences in a human VH framework. The CDR sequences may be situated, as an example, in the region of around amino acid residues 26-33; 51-58; and 97-116 for CDR1, CDR2 and CDR3, respectively, of the provided exemplary variable region sequences set forth in SEQ ID NOs: 7-8. It will be understood by one of ordinary skill in the art that the CDR sequences may be in different positions if a different framework sequence is selected, although generally the order of the sequences will remain the same.

In a particular embodiment, an anti-MUC1-C antibody comprises a CDR1 sequence of any one of SEQ ID NOs: 1 or 4. In a particular embodiment, the CDR1 sequence comprises SEQ ID NO: 1. In a particular embodiment, the CDR1 sequence comprises SEQ ID NO: 4.

In a particular embodiment, an anti-MUC1-C antibody comprises a CDR2 sequence of any one of SEQ ID NOs: 2 or 5. In a particular embodiment, the CDR2 sequence comprises SEQ ID NO: 2. In a particular embodiment, the CDR2 sequence comprises SEQ ID NO: 5.

In a particular embodiment, an anti-MUC1-C antibody comprises a CDR3 sequence of any one of SEQ ID NOs: 3 or 6. In a particular embodiment, the CDR3 sequence comprises SEQ ID NO: 3. In a particular embodiment, the CDR2 sequence comprises SEQ ID NO: 6.

In a further embodiment, an anti-MUC1-C heavy chain-only antibody comprises the CDR1 sequence of SEQ ID NO: 1; the CDR2 sequence of SEQ ID NO: 2; and the CDR3 sequence of SEQ ID NO: 3.

In a further embodiment, an anti-MUC1-C antibody comprises the CDR1 sequence of SEQ ID NO:4; the CDR2 sequence of SEQ ID NO: 5; and the CDR3 sequence of SEQ ID NO: 6.

In a further embodiment, an anti-MUC1-C antibody comprises any of the heavy chain variable region amino acid sequences of SEQ ID NOs: 7-8 (Table 3).

In a still further embodiment, an anti-MUC1-C antibody comprises the heavy chain variable region sequence of SEQ ID NO: 7.

In a still further embodiment, an anti-MUC1-C antibody comprises the heavy chain variable region sequence of SEQ ID NO: 8.

In some embodiments, a CDR sequence in an anti-MUC1-C antibody of the invention comprises one or two amino acid substitutions relative to a CDR1, CDR2 and/or CDR3 sequence or set of CDR1, CDR2 and CDR3 sequences in any one of SEQ ID NOs: 1-6 (Table 1; Table 2).

In some embodiments, an anti-MUC1-C antibody preferably comprises a heavy chain variable domain (VH) in which the CDR3 sequence has greater than or equal to 80%, such as at least 85%, at least 90%, at least 95%, or at least 99% sequence identity at the amino acid level to a CDR3 sequence of any one of the antibodies whose CDR3 sequences are provided in Table 1 or Table 2, and binds to MUC1 -C.

In some embodiments, an anti-MUC1-C antibody preferably comprises a heavy chain variable domain (VH) in which the full set of CDRs 1, 2, and 3 (combined) has greater than or equal to eighty-five percent (85%) sequence identity at the amino acid level to the CDRs 1, 2, and 3 (combined) of the antibodies whose CDR sequences are provided in Table 1 or Table 2, and binds to MUC1-C.

In some embodiments, an anti-MUC1-C antibody comprises a heavy chain variable region sequence with at least about 80% identity, at least 85% identity, at least 90% identity, at least 95% identity, at least 98% identity, or at least 99% identity to any of the heavy chain variable region sequences of SEQ ID NOs: 7-8 (shown in Table 3), and binds to MUC1-C.

In some embodiments, bispecific or multi-specific antibodies are provided, which may have any of the configurations discussed herein, including, without limitation, a bispecific three-chain antibody-like molecule (TCA). In some embodiments, a multi-specific antibody can comprise at least one heavy chain variable region having binding specificity for MUC1-C, and at least one heavy chain variable region having binding specificity for a protein other than MUC1-C. In some embodiments, a multi-specific antibody can comprise at least one heavy chain variable region that binds to MUC1-C, and at least one heavy chain variable region that binds to a protein other than MUC1-C. In some embodiments, a multi-specific antibody can comprise a heavy chain variable region comprising at least two antigen-binding domains, wherein each of the antigen-binding domains binds to MUC1-C. In some embodiments, a multi-specific antibody can comprise a heavy chain/light chain pair that binds to a first antigen (e.g., CD3), and a heavy chain from a heavy chain-only antibody. In certain embodiments, the heavy chain from the heavy chain-only antibody comprises an Fc portion comprising CH2 and/or CH3 and/or CH4 domains, in the absence of a CH1 domain. In one particular embodiment, a bispecific antibody comprises a heavy chain/light chain pair that binds to an antigen on an effector cell (e.g., a CD3 protein on a T-cell), and a heavy chain from a heavy chain-only antibody comprising an antigen-binding domain that binds to MUC1-C.

In some embodiments, a multi-specific antibody comprises a CD3-binding VH domain that is paired with a light chain variable domain. In certain embodiments, the light chain is a fixed light chain. In some embodiments, the CD3-binding VH domain comprises a CDR1 sequence of SEQ ID NO: 9, a CDR2 sequence of SEQ ID NO: 10, and a CDR3 sequence of SEQ ID NO: 11, in a human VH framework. In some embodiments, the CD3-binding VH domain comprises a CDR1 sequence of SEQ ID NO: 12, a CDR2 sequence of SEQ ID NO: 13, and a CDR3 sequence of SEQ ID NO: 14, in a human VH framework. In some embodiments, the fixed light chain comprises a CDR1 sequence of SEQ ID NO: 15, a CDR2 sequence of SEQ ID NO: 16, and a CDR3 sequence of SEQ ID NO: 17, in a human VL framework. Together, the CD3-binding VH domain and the light chain variable domain have binding affinity for CD3. In some embodiments, a CD3-binding VH domain comprises a heavy chain variable region sequence of SEQ ID NO: 18. In some embodiments, a CD3-binding VH domain comprises a heavy chain variable region sequence of SEQ ID NO: 19. In some embodiments, a CD3-binding VH domain comprises a sequence having at least about 80%, at least about 85%, at least about 90%, at least about 95%, or at least about 99% percent identity to the heavy chain variable region sequence of SEQ ID NO: 18 or 19. In some embodiments, a fixed light chain comprises a light chain variable region sequence of SEQ ID NO: 20. In some embodiments, a fixed light chain comprises a sequence having at least about 80%, at least about 85%, at least about 90%, at least about 95%, or at least about 99% percent identity to the heavy chain variable region sequence of SEQ ID NO: 20.

Multi-specific antibodies comprising the above-described CD3-binding VH domain and light chain variable domain have advantageous properties, for example, as described in published PCT application publication number WO2018/052503, the disclosure of which is incorporated by reference herein in its entirety. Any of the multi-specific antibodies and antigen-binding domains described herein, having binding affinity to MUC1-C, can be combined with the CD3-binding domains and fixed light chain domains described herein (see, e.g., Table 4 and Table 5), as well as additional sequences, such as those provided in Table 6 and Table 7, to generate multi-specific antibodies having binding affinity to one or more MUC1-C epitopes, as well as CD3.

TABLE 4
Anti-CD3 Heavy and Light Chain
CDR1, CDR2, CDR3 amino acid sequences.

| | SEQ_aa_CDR1 | SEQ_aa_CDR2 | SEQ_aa_CDR3 |
|---|---|---|---|
| Heavy | GFTFDDYA | ISWNSGSI | AKDSRGYGDYRLGGAY |
| Chain | (SEQ ID | (SEQ ID NO: 10) | (SEQ ID NO: 11) |
| (F2B) | NO: 9) | | |
| Heavy | GFTFHNYA | ISWNSGSI | AKDSRGYGDYSLGGAY |
| Chain | (SEQ ID | (SEQ ID NO: 13) | (SEQ ID NO: 14) |
| (F2F) | NO: 12) | | |
| Light | QSVSSN | GAS | QQYNNWPWT |
| Chain | (SEQ ID | (SEQ ID NO: 16) | (SEQ ID NO: 17) |
| | NO: 15) | | |

TABLE 5
Anti-CD3 heavy and light chain
variable region amino acid sequences.

| | |
|---|---|
| VH | EVQLVESGGGLVQPGRSLRLSCAASGFTFDDYAMHWV |
| (F2B) | RQAPGKGLEWVSGISWNSGSIGYADSVKGRFTISRDN |
| | AKNSLYLQMNSLRAEDTALYYCAKDSRGYGDYRLGGA |
| | YWGQGTLVTVSS (SEQ ID NO: 18) |
| VH | EVQLVESGGGLVQPGRSLRLSCAASGFTFHNYAMHWV |
| (F2F) | RQAPGKGLEWVSGISWNSGSIGYADSVKGRFTISRDN |
| | AKNSLYLQMNSLRAEDTALYYCAKDSRGYGDYSLGGA |
| | YWGQGTLVTVSS (SEQ ID NO: 19) |
| VL | EIVMTQSPATLSVSPGERATLSCRASQSVSSNLAWYQ |
| | QKPGQAPRLLIYGASTRATGIPARFSGSGSGTEFTLT |
| | ISSLQSEDFAVYYCQQYNNWPWTFGQGTKVEIK |
| | (SEQ ID NO: 20) |

TABLE 6
Human IgG1 and IgG4 Fc region sequences.

| | |
|---|---|
| Human IgG1 | ASTKGPSVFPLAPSSKSTSGGTAALGCLVKDYFPEPVTVS |
| (UniProt | WNSGALTSGVHTFPAVLQSSGLYSLSSVVTVPSSSLGTQT |
| No. | YICNVNHKPSNTKVDKKVEPKSCDKTHTCPPCPAPELLGG |
| P01857) | PSVFLFPPKPKDTLMISRTPEVTCVVVDVSHEDPEVKFNW |
| | YVDGVEVHNAKTKPREEQYNSTYRVVSVLTVLHQDWLNGK |
| | EYKCKVSNKALPAPIEKTISKAKGQPREPQVYTLPPSRDE |
| | LTKNQVSLTCLVKGFYPSDIAVEWESNGQPENNYKTTPPV |
| | LDSDGSFFLYSKLTVDKSRWQQGNVFSCSVMHEALHNHYT |
| | QKSLSLSPGK (SEQ ID NO: 21) |
| Human IgG4 | ASTKGPSVFPLAPCSRSTSESTAALGCLVKDYFPEPVTVS |
| (UniProt | WNSGALTSGVHTFPAVLQSSGLYSLSSVVTVPSSSLGTKT |
| No. | YTCNVDHKPSNTKVDKRVESKYGPPCPSCPAPEFLGGPSV |
| P01861) | FLFPPKPKDTLMISRTPEVTCVVVDVSQEDPEVQFNWYVD |
| | GVEVHNAKTKPREEQFNSTYRVVSVLTVLHQDWLNGKEYK |
| | CKVSNKGLPSSIEKTISKAKGQPREPQVYTLPPSQEEMTK |
| | NQVSLTCLVKGFYPSDIAVEWESNGQPENNYKTTPPVLDS |
| | DGSFFLYSRLTVDKSRWQEGNVFSCSVMHEALHNHYTQKS |
| | LSLSLGK (SEQ ID NO: 22) |
| Human IgG1 | ASTKGPSVFPLAPSSKSTSGGTAALGCLVKDYFPEPVTVS |
| with | WNSGALTSGVHTFPAVLQSSGLYSLSSVVTVPSSSLGTQT |
| silencing | YICNVNHKPSNTKVDKKVEPKSCDKTHTCPPCPAPEAAGG |
| mutations | PSVFLFPPKPKDTLMISRTPEVTCVVVDVSHEDPEVKFNW |
| (Fc region) | YVDGVEVHNAKTKPREEQYNSTYRVVSVLTVLHQDWLNGK |
| | EYKCKVSNKALPAPIEKTISKAKGQPREPQVYTLPPSREE |
| | MTKNQVSLTCLVKGFYPSDIAVEWESNGQPENNYKTTPPV |
| | LDSDGSFFLYSKLTVDKSRWQQGNVFSCSVMHEALHNHYT |
| | QKSLSLSPGK (SEQ ID NO: 23) |
| Human IgG4 | ASTKGPSVFPLAPCSRSTSESTAALGCLVKDYFPEPVTVS |
| with | WNSGALTSGVHTFPAVLQSSGLYSLSSVVTVPSSSLGTKT |
| silencing | YTCNVDHKPSNTKVDKRVESKYGPPCPPCPAPEAAGGPSV |
| mutations | FLFPPKPKDTLMISRTPEVTCVVVDVSQEDPEVQFNWYVD |
| (Fc region) | GVEVHNAKTKPREEQFNSTYRVVSVLTVLHQDWLNGKEYK |
| | CKVSNKGLPSSIEKTISKAKGQPREPQVYTLPPSQEEMTK |
| | NQVSLTCLVKGFYPSDIAVEWESNGQPENNYKTTPPVLDS |
| | DGSFFLYSRLTVDKSRWQEGNVFSCSVMHEALHNHYTQKS |
| | LSLSLGK (SEQ ID NO: 24) |

TABLE 7
additional sequences.

| | |
|---|---|
| Anti-CD3 light | RTVAAPSVFIFPPSDEQLKSGTASVVCLLNNFYPREAKVQWKVDNAL |
| chain constant | QSGNSQESVTEQDSKDSTYSLSSTLTLSKADYEKHKVYACEVTHQGL |
| region sequence | SSPVTKSFNRGEC (SEQ ID NO: 25) |
| (kappa light chain) | |
| Anti-CD3 heavy | EVQLVESGGGLVQPGRSLRLSCAASGFTFDDYAMHWVRQAPGKGLE |
| chain sequence | WVSGISWNSGSIGYADSVKGRFTISRDNAKNSLYLQMNSLRAEDTAL |
| (VH + wt IgG1 Fc) | YYCAKDSRGYGDYRLGGAYWGQGTLVTVSSASTKGPSVFPLAPSSKS |
| | TSGGTAALGCLVKDYFPEPVTVSWNSGALTSGVHTFPAVLQSSGLYS |
| | LSSVVTVPSSSLGTQTYICNVNHKPSNTKVDKKVEPKSCDKTHTCPPC |
| | PAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRW |
| | QQGNVFSCSVMHEALHNHYTQKSLSLSPGK (SEQ ID NO: 26) |
| Anti-CD3 heavy | EVQLVESGGGLVQPGRSLRLSCAASGFTFDDYAMHWVRQAPGKGLE |
| chain sequence (with | WVSGISWNSGSIGYADSVKGRFTISRDNAKNSLYLQMNSLRAEDTAL |
| silenced IgG1 Fc) | YYCAKDSRGYGDYRLGGAYWGQGTLVTVSSASTKGPSVFPLAPSSKS |
| | TSGGTAALGCLVKDYFPEPVTVSWNSGALTSGVHTFPAVLQSSGLYS |
| | LSSVVTVPSSSLGTQTYICNVNHKPSNTKVDKKVEPKSCDKTHTCPPC |
| | PAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCK |
| | VSNKALPAPIEKTISKAKGQPREPQVYTLPPSREEMTKNQVSLTCLVK |
| | GFYPSDIAVEWESNGQPENNYKTTPPVLDSDGSFFLYSKLTVDKSRW |
| | QQGNVFSCSVMHEALHNHYTQKSLSLSPGK (SEQ ID NO: 27) |
| Anti-CD3 heavy | EVQLVESGGGLVQPGRSLRLSCAASGFTFDDYAMHWVRQAPGKGLE |
| chain constant | WVSGISWNSGSIGYADSVKGRFTISRDNAKNSLYLQMNSLRAEDTAL |
| region sequence | YYCAKDSRGYGDYRLGGAYWGQGTLVTVSSASTKGPSVFPLAPCSRS |
| (with wt IgG4 Fc) | TSESTAALGCLVKDYFPEPVTVSWNSGALTSGVHTFPAVLQSSGLYSL |
| | SSVVTVPSSSLGTKTYTCNVDHKPSNTKVDKRVESKYGPPCPSCPAPE |
| | FLGGPSVFLFPPKPKDTLMISRTPEVTCVVVDVSQEDPEVQFNWYVDG |
| | VEVHNAKTKPREEQFNSTYRVVSVLTVLHQDWLNGKEYKCKVSNKG |
| | LPSSIEKTISKAKGQPREPQVYTLPPSQEEMTKNQVSLTCLVKGFYPSD |
| | IAVEWESNGQPENNYKTTPPVLDSDGSFFLYSRLTVDKSRWQEGNVF |
| | SCSVMHEALHNHYTQKSLSLSLGK (SEQ ID NO: 28) |
| Anti-CD3 heavy | EVQLVESGGGLVQPGRSLRLSCAASGFTFDDYAMHWVRQAPGKGLE |
| chain constant | WVSGISWNSGSIGYADSVKGRFTISRDNAKNSLYLQMNSLRAEDTAL |
| region sequence | YYCAKDSRGYGDYRLGGAYWGQGTLVTVSSASTKGPSVFPLAPCSRS |
| (with silenced IgG4 | TSESTAALGCLVKDYFPEPVTVSWNSGALTSGVHTFPAVLQSSGLYSL |
| Fc) | SSVVTVPSSSLGTKTYTCNVDHKPSNTKVDKRVESKYGPPCPPCPAPE |
| | AAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNV |
| | FSCSVMHEALHNHYTQKSLSLSLGK (SEQ ID NO: 29) |
| Silenced IgG4 | ESKYGPPCP [symbol] CPAPE [symbol] GGPSVFLFPPKPKDTLMISRTPEVTCVVVD |
| (hinge-CH2- | VSQEDPEVQFNWYVDGVEVHNAKTKPREEQFNSTYRVVSVLTVLHQ |
| CH3; hole (S228P, | DWLNGKEYKCKVSNKGLPSSIEKTISKAKGQPREPQVYTLPPSQEEMT |
| F234A, L235A; | KNQVSL [symbol] C [symbol] VKGFYPSDIAVEWESNGQPENNYKTTPPVLDSDGSFFL |
| T366S, L368A, | [symbol] SRLTVDKSRWQEGNVFSCSVMHEALHNHYTQKSLSLSLGK |
| Y407V)) | (SEQ ID NO: 30) |
| Silenced IgG4 | ESKYGPPCP [symbol] CPAPE [symbol] GGPSVFLFPPKPKDTLMISRTPEVTCVVVD |
| (hinge-CH2- | VSQEDPEVQFNWYVDGVEVHNAKTKPREEQFNSTYRVVSVLTVLHQ |
| CH3; knob (S228P, | DWLNGKEYKCKVSNKGLPSSIEKTISKAKGQPREPQVYTLPPSQEEMT |
| F234A, L235A; | KNQVSL [symbol] CLVKGFYPSDIAVEWESNGQPENNYKTTPPVLDSDGSFF |
| T366W)) | LYSRLTVDKSRWQEGNVFSCSVMHEALHNHYTQKSLSLSLGK (SEQ |
| | ID NO: 31) |
| Anti-CD3 full length | EIVMTQSPATLSVSPGERATLSCRASQSVSSNLAWYQQKPGQAPRLLI |
| light chain (VL + | YGASTRATGIPARFSGSGSGTEFTLTISSLQSEDFAVYYCQQYNNWPW |
| kappa CL) | TFGQGTKVEIKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC (SEQ ID NO: 32) |
| Anti-CD3 (F2B) full | EVQLVESGGGLVQPGRSLRLSCAASGFTFDDYAMHWVRQAPGKGLE |
| length heavy chain | WVSGISWNSGSIGYADSVKGRFTISRDNAKNSLYLQMNSLRAEDTAL |
| (VH + silenced IgG4 | YYCAKDSRGYGDYRLGGAYWGQGTLVTVSSASTKGPSVFPLAPCSRS |
| Fc + knob (S228P, | TSESTAALGCLVKDYFPEPVTVSWNSGALTSGVHTFPAVLQSSGLYSL |
| F234A, L235A; | SSVVTVPSSSLGTKTYTCNVDHKPSNTKVDKRVESKYGPPCPPCPAPE |
| T366W)) | AAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNK |
| | GLPSSIEKTISKAKGQPREPQVYTLPPSQEEMTKNQVSLWCLVKGFYP |
| | SDIAVEWESNGQPENNYKTTPPVLDSDGSFFLYSRLTVDKSRWQEGN |
| | VFSCSVMHEALHNHYTQKSLSLSLGK (SEQ ID NO: 33) |
| Anti-CD3 (F2F) full | EVQLVESGGGLVQPGRSLRLSCAASGFTFHNYAMHWVRQAPGKGLE |
| length heavy chain | WVSGISWNSGSIGYADSVKGRFTISRDNAKNSLYLQMNSLRAEDTAL |
| (VH + silenced IgG4 | YYCAKDSRGYGDYSLGGAYWGQGTLVTVSSASTKGPSVFPLAPCSRS |
| Fc + knob (S228P, | TSESTAALGCLVKDYFPEPVTVSWNSGALTSGVHTFPAVLQSSGLYSL |
| F234A, L235A; | SSVVTVPSSSLGTKTYTCNVDHKPSNTKVDKRVESKYGPPCPPCPAPE |
| T366W)) | AAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNK |
| | GLPSSIEKTISKAKGQPREPQVYTLPPSQEEMTKNQVSLWCLVKGFYP |
| | SDIAVEWESNGQPENNYKTTPPVLDSDGSFFLYSRLTVDKSRWQEGN |
| | VFSCSVMHEALHNHYTQKSLSLSLGK (SEQ ID NO: 42) |
| Anti-MUC1-C | EVQLVESGGGLVQPGGSLRLSCTASGFAFSGNSMNWVRQAPGKGLE |
| monovalent heavy | WVAFITSSGRSIKYADSVKGRFTISRDNAKNSLYLQMNTLRDEDTALY |
| chain (clone ID | YCATGGTGTSLFDYRGQGTLVTVSSESKYGPPCPPCPAPEAAGGPSVF |
| 375747) + silenced | LFPPKPKDTLMISRTPEVTCVVVDVSQEDPEVQFNWYVDGVEVHNAK |
| IgG4 Fc, hole | TKPREEQFNSTYRVVSVLTVLHQDWLNGKEYKCKVSNKGLPSSIEKTI |
| (S228P, F234A, | SKAKGQPREPQVYTLPPSQEEMTKNQVSLSCAVKGFYPSDIAVEWES |
| L235A, T366S, | NGQPENNYKTTPPVLDSDGSFFLVSRLTVDKSRWQEGNVFSCSVMHE |
| L368A, Y407V | ALHNHYTQKSLSLSLGK (SEQ ID NO: 34) |
| Anti-MUC1-C | EVQLVESGGGLVQPGGSLRLSCTASGFAFSGNSMNWVRQAPGKGLE |
| bivalent heavy chain | WVAFITSSGRSIKYADSVKGRFTISRDNAKNSLYLQMNTLRDEDTALY |
| (clone ID 375747) + | YCATGGTGTSLFDYRGQGTLVTVSSGGGGSGGGGSEVQLVESGGGL |
| silenced IgG4 Fc, | VQPGGSLRLSCTASGFAFSGNSMNWVRQAPGKGLEWVAFITSSGRSI |
| hole (S228P, F234A, | KYADSVKGRFTISRDNAKNSLYLQMNTLRDEDTALYYCATGGTGTSL |
| L235A, T366S, | FDYRGQGTLVTVSSESKYGPPCPPCPAPEAAGGPSVFLFPPKPKDTLMI |
| L368A, Y407V | SRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREP |
| | QVYTLPPSQEEMTKNQVSLSCAVKGFYPSDIAVEWESNGQPENNYKT |
| | TPPVLDSDGSFFLVSRLTVDKSRWQEGNVFSCSVMHEALHNHYTQKS |
| | LSLSLGK (SEQ ID NO: 35) |
| Anti-MUC1-C | EVQLVESGGGLVQPGGSLRLSCAASGFTFSSHSMNWVRQAPGKGLE |
| monovalent heavy | WVSFISSSSNIKKYADSVKGRFTISRDNAKNSLFLQMNSLRDEDTAVY |
| chain (clone ID | YCATGGTGITVLDYRGQGTLVTVSSESKYGPPCPPCPAPEAAGGPSVF |
| 375505) + silenced | LFPPKPKDTLMISRTPEVTCVVVDVSQEDPEVQFNWYVDGVEVHNAK |
| IgG4 Fc, hole | TKPREEQFNSTYRVVSVLTVLHQDWLNGKEYKCKVSNKGLPSSIEKTI |
| (S228P, F234A, | SKAKGQPREPQVYTLPPSQEEMTKNQVSLSCAVKGFYPSDIAVEWES |
| L235A, T366S, | NGQPENNYKTTPPVLDSDGSFFLVSRLTVDKSRWQEGNVFSCSVMHE |
| L368A, Y407V | ALHNHYTQKSLSLSLGK (SEQ ID NO: 36) |
| Anti-MUC1-C | EVQLVESGGGLVQPGGSLRLSCAASGFTFSSHSMNWVRQAPGKGLE |
| bivalent havy chain | WVSFISSSSNIKKYADSVKGRFTISRDNAKNSLFLQMNSLRDEDTAVY |
| (clone ID 375505) + | YCATGGTGITVLDYRGQGTLVTVSSGGGGSGGGGSEVQLVESGGGLV |
| silenced IgG4 Fc, | QPGGSLRLSCAASGFTFSSHSMNWVRQAPGKGLEWVSFISSSSNIKKY |
| hole (S228P, F234A, | ADSVKGRFTISRDNAKNSLFLQMNSLRDEDTAVYYCATGGTGITVLD |
| L235A, T366S, | YRGQGTLVTVSSESKYGPPCPPCPAPEAAGGPSVFLFPPKPKDTLMISR |
| L368A, Y407V | TPEVTCVVVDVSQEDPEVQFNWYVDGVEVHNAKTKPREEQFNSTYR |
| | VVSVLTVLHQDWLNGKEYKCKVSNKGLPSSIEKTISKAKGQPREPQV |
| | YTLPPSQEEMTKNQVSLSCAVKGFYPSDIAVEWESNGQPENNYKTTPP |
| | VLDSDGSFFLVSRLTVDKSRWQEGNVFSCSVMHEALHNHYTQKSLSL |
| | SLGK (SEQ ID NO: 37) |
| Anti-MUC1-C | EVQLVESGGGLVQPGGSLRLSCTASGFAFSGNSMNWVRQAPGKGLE |
| bivalent havy chain | WVAFITSSGRSIKYADSVKGRFTISRDNAKNSLYLQMNTLRDEDTALY |
| (clone ID 375747 × | YCATGGTGTSLFDYRGQGTLVTVSSGGGGSGGGGSEVQLVESGGGL |
| clone ID 375505) + | VQPGGSLRLSCAASGFTFSSHSMNWVRQAPGKGLEWVSFISSSSNIKK |
| silenced IgG4 Fc, | YADSVKGRFTISRDNAKNSLFLQMNSLRDEDTAVYYCATGGTGITVL |
| hole (S228P, F234A, | DYRGQGTLVTVSSESKYGPPCPPCPAPEAAGGPSVFLFPPKPKDTLMIS |
| L235A, T366S, | RTPEVTCVVVDVSQEDPEVQFNWYVDGVEVHNAKTKPREEQFNSTY |
| L368A, Y407V | RVVSVLTVLHQDWLNGKEYKCKVSNKGLPSSIEKTISKAKGQPREPQ |
| | VYTLPPSQEEMTKNQVSLSCAVKGFYPSDIAVEWESNGQPENNYKTT |
| | PPVLDSDGSFFLVSRLTVDKSRWQEGNVFSCSVMHEALHNHYTQKSL |
| | SLSLGK (SEQ ID NO: 38) |
| Anti-MUC1-C | EVQLVESGGGLVQPGGSLRLSCAASGFTFSSHSMNWVRQAPGKGLE |
| bivalent heavy chain | WVSFISSSSNIKKYADSVKGRFTISRDNAKNSLFLQMNSLRDEDTAVY |
| (clone ID 375505 × | YCATGGTGITVLDYRGQGTLVTVSSGGGGSGGGGSEVQLVESGGGLV |
| clone ID 375747) + | QPGGSLRLSCTASGFAFSGNSMNWVRQAPGKGLEWVAFITSSGRSIK |
| silenced IgG4 Fc, | YADSVKGRFTISRDNAKNSLYLQMNTLRDEDTALYYCATGGTGTSLF |
| hole (S228P, F234A, | DYRGQGTLVTVSSESKYGPPCPPCPAPEAAGGPSVFLFPPKPKDTLMIS |
| L235A, T366S, | RTPEVTCVVVDVSQEDPEVQFNWYVDGVEVHNAKTKPREEQFNSTY |
| L368A, Y407V | RVVSVLTVLHQDWLNGKEYKCKVSNKGLPSSIEKTISKAKGQPREPQ |
| | VYTLPPSQEEMTKNQVSLSCAVKGFYPSDIAVEWESNGQPENNYKTT |
| | PPVLDSDGSFFLVSRLTVDKSRWQEGNVFSCSVMHEALHNHYTQKSL |
| | SLSLGK (SEQ ID NO: 39) |

In some embodiments, bispecific or multi-specific antibodies are provided, which may have any of the configurations discussed herein, including, without limitation, a bispecific three-chain antibody like molecule (TCA). In some embodiments, a bispecific antibody can comprise at least one heavy chain variable region that binds to MUC1-C, and at least one heavy chain variable region that binds to a protein other than MUC1-C. In some embodiments, a bispecific antibody can comprise a heavy chain/light chain pair that binds to a first antigen, and a heavy chain from a heavy chain-only antibody, comprising an Fc portion comprising CH2 and/or CH3 and/or CH4 domains, in the absence of a CH1 domain, and an antigen binding domain that binds an epitope of a second antigen or a different epitope of the first antigen. In one particular embodiment, a bispecific antibody comprises a heavy chain/light chain pair that binds to an antigen on an effector cell (e.g., a CD3 protein on a T-cell), and a heavy chain from a heavy chain-only antibody comprising an antigen-binding domain that binds to MUC1-C.

In some embodiments, where an antibody of the invention is a bispecific antibody, one arm of the antibody (one binding moiety, or one binding unit) is specific for human MUC1-C, while the other arm may be specific for target cells, tumor-associated antigens, targeting antigens, e.g., integrins, etc., pathogen antigens, checkpoint proteins, and the like. Target cells specifically include cancer cells, including, without limitation, cells associated with solid tumors and/or hematological malignancies characterized by the expression of MUC1-C. In some embodiments, one arm of the antibody (one binding moiety, or one binding unit) is specific for human MUC1-C, while the other arm is specific for CD3.

In some embodiments, an antibody comprises an anti-CD3 light chain polypeptide comprising the sequence of SEQ ID NO: 32, an anti-CD3 heavy chain polypeptide comprising the sequence of SEQ ID NO: 18 or 19, and an anti-MUC1-C heavy chain polypeptide comprising the sequence of SEQ ID NO: 7 or 8, in a monovalent or bivalent configuration, linked to the sequence of any one of SEQ ID NOs: 30 or 31. These sequences can be combined in various ways to produce a bispecific antibody of a desired IgG subclass, e.g., IgG1, IgG4, silenced IgG1, silenced IgG4. In one preferred embodiment, an antibody is a TCA comprising a first polypeptide comprising SEQ ID NO: 32, a second polypeptide comprising SEQ ID NO: 33, and a third polypeptide comprising SEQ ID NO: 34, 35, 36, 37, 38 or 39.

Various formats of multi-specific antibodies are within the ambit of the invention, including, without limitation, single chain polypeptides, two chain polypeptides, three chain polypeptides, four chain polypeptides, and multiples thereof. The multi-specific antibodies herein specifically include T-cell multi-specific (e.g., bispecific) antibodies binding to MUC1-C and CD3 (anti-MUC1-C x anti-CD3 antibodies). Such antibodies induce potent T-cell mediated killing of cells expressing MUC1-C.

### Preparation of Anti-MUCI-C Antibodies

The antibodies of the present invention can be prepared by methods known in the art. In a preferred embodiment, the antibodies herein are produced by transgenic animals, including transgenic mice and rats, preferably rats, in which the endogenous immunoglobulin genes are knocked out or disabled. In a preferred embodiment, the heavy chain antibodies herein are produced in a UniRat™ UniRats™ have their endogenous immunoglobulin genes silenced and use a human immunoglobulin heavy-chain translocus to express a diverse, naturally optimized repertoire of fully human HCAbs. While endogenous immunoglobulin loci in rats can be knocked out or silenced using a variety of technologies, in UniRat™ the zinc-finger (endo)nuclease (ZNF) technology was used to inactivate the endogenous rat heavy chain J-locus, light chain Cκ locus and light chain Cλ locus. ZNF constructs for microinjection into oocytes can produce IgH and IgL knock out (KO) lines. For details see, e.g., Geurts et al., 2009, Science 325:433. Characterization of Ig heavy chain knockout rats has been reported by Menoret et al., 2010, Eur. J. Immunol. 40:2932-2941. Advantages of the ZNF technology are that non-homologous end joining to silence a gene or locus via deletions up to several kb can also provide a target site for homologous integration (Cui et al., 2011, Nat Biotechnol 29:64-67). Human heavy chain antibodies produced in a UniRat™ are called UniAbs™ and can bind epitopes that cannot be attacked with conventional antibodies. Their high specificity, affinity, and small size make them ideal for mono- and poly-specific applications.

In addition to UniAbs™, specifically included herein are heavy chain-only antibodies lacking the camelid VHH framework and mutations, and their functional VH regions. Such heavy chain-only antibodies can, for example, be produced in transgenic rats or mice which comprise fully human heavy chain-only gene loci as described, e.g., in WO2006/008548, but other transgenic mammals, such as rabbit, guinea pig, rat can also be used, rats and mice being preferred. Heavy chain-only antibodies, including their VHH or VH functional fragments, can also be produced by recombinant DNA technology, by expression of the encoding nucleic acid in a suitable eukaryotic or prokaryotic host, including, for example, mammalian cells (e.g., CHO cells), E. coli or yeast.

Domains of heavy chain-only antibodies combine advantages of antibodies and small molecule drugs: can be mono- or multi-valent; have low toxicity; and are cost-effective to manufacture. Due to their small size, these domains are easy to administer, including oral or topical administration, are characterized by high stability, including gastrointestinal stability; and their half-life can be tailored to the desired use or indication. In addition, VH and VHH domains of HCAbs can be manufactured in a cost-effective manner.

In a particular embodiment, the heavy chain antibodies of the present invention, including UniAbs™, have the native amino acid residue at the first position of the FR4 region (amino acid position 101 according to the Kabat numbering system), substituted by another amino acid residue, which is capable of disrupting a surface-exposed hydrophobic patch comprising or associated with the native amino acid residue at that position. Such hydrophobic patches are normally buried in the interface with the antibody light chain constant region but become surface exposed in HCAbs and are, at least partially, for the unwanted aggregation and light chain association of HCAbs. The substituted amino acid residue preferably is charged, and more preferably is positively charged, such as lysine (Lys, K), arginine (Arg, R) or histidine (His, H), preferably arginine (R). In a preferred embodiment the heavy chain-only antibodies derived from the transgenic animals contain a Trp to Arg mutation at position 101. The resultant HCAbs preferably have high antigen-binding affinity and solubility under physiological conditions in the absence of aggregation.

As part of the present invention, human IgG anti-MUC1-C heavy chain antibodies with unique sequences from UniRat™ animals (UniAb™) were identified that bind to human MUC1-C in ELISA protein and cell-binding assays. The identified heavy chain variable region (VH) sequences are positive for human MUC1-C protein binding and/or for binding to MUC1-C+cells, and are all negative for binding to cells that do not express MUC1-C.

Heavy chain antibodies binding to non-overlapping epitopes on a MUC1-C protein, e.g., UniAbs™ can be identified by competition binding assays, such as enzyme-linked immunoassays (ELISA assays) or flow cytometric competitive binding assays. For example, one can use competition between known antibodies binding to the target antigen and the antibody of interest. By using this approach, one can divide a set of antibodies into those that compete with the reference antibody and those that do not. The non-competing antibodies are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc). For further details see the examples herein.

Typically, an antibody “competes” with a reference antibody if it causes about 15-100% reduction in the binding of the reference antibody to the target antigen, as determined by standard techniques, such as by the competition binding assays described above. In various embodiments, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

### Pharmaceutical Compositions, Uses and Methods of Treatment

It is another aspect of the present invention to provide pharmaceutical compositions comprising one or more antibodies of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one embodiment, a pharmaceutical composition comprises a heavy chain antibody (e.g., UniAb™) that binds to MUC1-C. In another embodiment, a pharmaceutical composition comprises a multi-specific (including bispecific) heavy chain antibody (e.g., UniAb™) with binding specificity for two or more non-overlapping epitopes on a MUC1-C protein. In a preferred embodiment, a pharmaceutical composition comprises a multi-specific (including bispecific and TCA) heavy chain antibody (e.g., UniAb™) with binding specificity to MUC1-C and with binding specificity to a binding target on an effector cell (e.g., a binding target on a T-cell, such as, e.g., a CD3 protein on a T-cell). In a preferred embodiment, a pharmaceutical composition comprises a multi-specific (including bispecific and TCA) heavy chain antibody (e.g., UniAb™) that binds to MUC1-C and that binds to a binding target on an effector cell (e.g., a binding target on a T-cell, such as, e.g., a CD3 protein on a T-cell).

Pharmaceutical compositions of the antibodies used in accordance with the present invention are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The antibodies herein can be administered by intravenous injection or infusion or subcutaneously. For injection administration, the antibodies herein can be formulated in aqueous solutions, preferably in physiologically-compatible buffers to reduce discomfort at the site of injection. The solution can contain carriers, excipients, or stabilizers as discussed above. Alternatively, antibodies can be in lyophilized form for constitution with a suitable vehicle, e.g., sterile pyrogen-free water, before use.

Antibody formulations are disclosed, for example, in U.S. Pat. No. 9,034,324. Similar formulations can be used for the heavy chain antibodies, including UniAbs™, of the present invention. Subcutaneous antibody formulations are described, for example, in US20160355591 and US20160166689.

### Methods of Use

The anti-MUC1-C antibodies and pharmaceutical compositions described herein can be used for the treatment of diseases and conditions characterized by the expression of MUC1-C, including, without limitation, the conditions and diseases described further herein.

Mucin 1 (MUC1) is a heavily glycosylated, single pass type I transmembrane protein. The N-terminal subunit (MUC1-N) and C-terminal subunit (MUC1-C) form a stable heterodimeric complex. MUC1 is highly polymorphic, with greater than 90 isoforms, differing in the number of tandem repeats in the VNTR (variable number tandem repeat) region of the N-terminal subunit. Mucins line the apical surface of epithelial cells in the lungs, stomach, mammary glands, intestines, and several other organs. In healthy tissues, mucins protect the body from infection. Aberrantly glycosylated MUC1 is overexpressed in human epithelial cancers and apical polarity is lost in tumor cells (Sousa et al. 2016, PMC: 4998183, Nath and Mukherjee, 2014, PMID: 5500204). MUC1 can be cleaved by proteases and cleaved MUC1-N is shed from the cell and can trigger inflammation. The non-shed oncogenic MUC1-C subunit is short, containing a 58 amino acid membrane proximal extracellular domain that shows promise as a target for antibody drug conjugates, monoclonal antibodies and CAR-T therapies (Panchamoorthy et al., 2018, PMC: 6124453; Kufe, 2009, PMC: 2951677).

In one aspect, the anti-MUC1-C antibodies (e.g., UniAbs™) and pharmaceutical compositions herein can be used to treat disorders characterized by the expression of MUC1-C, including, without limitation, the diseases and disorders described further herein.

The anti-MUC1-C heavy chain-only antibodies (UniAbs) of the present invention can be used to develop therapeutic agents for the treatment of cancers, including solid tumors and hematological malignancies, such as those described further herein. Solid tumors include cancers of epithelial origin, i.e., carcinomas, including adenocarcinomas and squamous cell carcinomas. Non-limiting examples of carcinomas include: breast, non-small cell lung (NSCL), small cell lung (SSC), mesothelioma, renal cell, colorectal, ovarian, head and neck squamous cell, nasopharyngeal, gastric, prostatic, pancreatic, esophageal, and cervical carcinomas. Hematological malignancies include, without limitation, myelomas, leukemias, and lymphomas. Non-limiting examples of hematological malignancies include multiple myeloma and chronic myeloid leukemia (CML). Although some monoclonal antibodies have shown promise for treating these diseases, consistent clinical efficacy has not yet been conclusively demonstrated. There is therefore a great need for new therapies, including immunotherapies, for these cancers.

In one embodiment, the antibodies herein can be in the form of heavy chain-only anti-MUC1-C antibody-CAR structures, i.e., heavy chain-only anti-MUC1-C antibody-CAR-transduced T-cell structures. FIG. 4 is a schematic illustration of a CAR-T structure comprising an anti-MUC1-C extracellular binding domain comprising a heavy chain variable region (VH) sequence in accordance with embodiments of the invention.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage the agent may range from about 0.0001 to 100 mg/kg, and more usually 0.01 to 5 mg/kg, of the host body weight. For example, dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent, e.g., a chemotherapeutic drug, etc. Kits typically include a label indicating the intended use of the contents of the kit. The term “label” as used herein includes any writing, or recorded material supplied on or with a kit, or which otherwise accompanies a kit.

The invention now being fully described, it will be apparent to one of ordinary skill in the art that various changes and modifications can be made without departing from the spirit or scope of the invention.

### EXAMPLES

### Example 1

### Binding to MUC1-C+ Raji Cells

Binding to MUC1-C-positive Raji cells was assessed by flow cytometry. Briefly, 50,000 target cells were stained with a dilution series of purified UniAbs™ for 30 minutes at 4° C. Following incubation, the cells were washed twice with flow cytometry buffer (1X PBS, 1% BSA, 0.1% NaN 3) and stained with goat F(ab′)₂ anti-human IgG conjugated to R-phycoerythrin (PE) (Southern Biotech, cat. #2042-09) to detect cell-bound antibodies. After a 20-minute incubation at 4° C., the cells were washed twice with flow cytometry buffer and the mean fluorescence intensity (MFI) was measured by flow cytometry. The MFI of cells stained with secondary antibody alone was used for determination of background signal, and binding of each antibody was converted to fold over background.

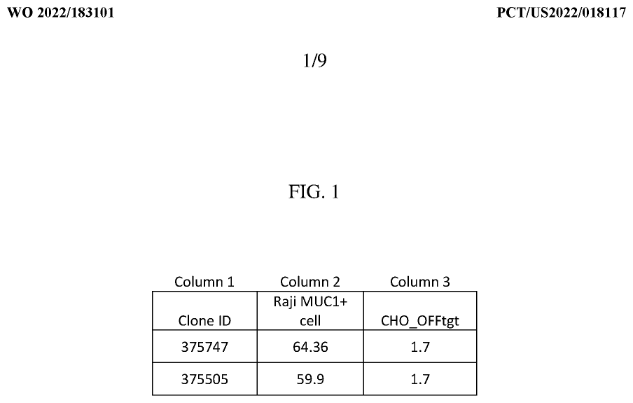

The results are provided in FIG. 1, which summarizes the target binding activity of the indicated anti-MUC1-C antibodies. Column 1 indicates the clone ID of the HCAb. Column 2 indicates binding to Raji cells measured as fold over background MFI signal. Column 3 indicates binding to CHO cells that do not express MUC1-C protein (negative control) measured as fold over background MFI signal.

### Example 2

### Binding to MUC1-C+ Raji Cells

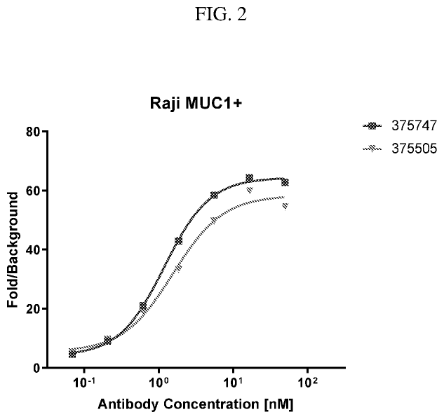

Cell-binding dose curves were performed on Raji MUC1-C+cells, as described in Example 1. The antibodies were tested at a starting concentration of 150 nM followed by 3-fold serial dilutions for an 8-point dose curve. PE mean fluorescence intensity was plotted as a fold over background (cells incubated with secondary detection antibody only). The results are provided in FIG. 2.

### Example 3

### Cell Binding EC50 Values on MUC1-C+ Raji Cells

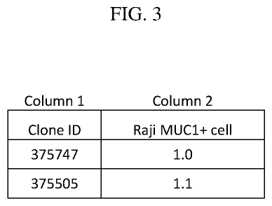

For determining cell binding EC50 values, cell binding dose curves were performed on Raji cells that express MUC1-C, as described above. The antibodies were tested at a starting dose of 150 nM, followed by 3-fold serial dilutions for an 8-point dose curve. The transformed data was plotted as an xy-graph using a non-linear regression curve fit (available in GraphPad Prism 8.4.3) to obtain the EC50s (nM). The results are provided in FIG. 3 (column 2 provides the EC50 values, in units of nM).

### Example 4

### CAR-T-Mediated T-Cell Activation by Human Tumor Cells

CAR-T cell activity was measured by transfecting Jurkat T lymphocyte cells with an anti-MUC1-C CAR and a 6x NFAT TK nano luciferase reporter. Transfected Jurkats were co-cultured for 24 hours with MUC1-C+Raji cells stably transfected to express human MUC1-C, or MUC1-C-negative Raji cells. Luciferase activity was measured using the Promega Nano-Glo Luciferase Assay System (catalog # N1110) and data were normalized to co-culture containing the CAR transfected Jurkat and MUC1-C negative Raji cell lines. Statistical significance was determined using an unpaired, two-tailed t-test. The results are provided in FIG. 4, Panels B and C.

FIG. 4, Panel A, is a schematic illustration of a CAR-T structure comprising an anti-MUC1-C extracellular binding domain comprising an antibody sequence in accordance with aspects of the invention. Panel B depicts T-cell activity of Jurkats transfected an anti-MUC1-C 375747 CAR with Raji-MUC1-C+(***p=0.0009). Panel C depicts T-cell activity of Jurkats transfected with an anti-MUC1-C 375505 CAR with Raji-MUC1-C+ (**p=0.0019). These results demonstrate that T-cell activation was specific to MUC1-C target binding, as co-culture with the MUC1-C-negative Raji cells, or incubation of the transfected Jurkats alone, did not result in appreciable luciferase reporter signal.

### Example 5

### Tumor Control by CAR-T-Mediated T-Cell Activation Assessment in vivo

A pre-clinical evaluation of tumor growth was assessed for antibody constructs corresponding to Clone ID Nos. 375505 and 375747 using a murine xenograft model of a triple-negative breast cancer (TNBC). A GFP- and luciferase-expressing MDA-MB-468 cell line (MDA-MB-468.1ucGFP; 5×10\hat{ }6 cells) was injected subcutaneously into NOD.Cg-Prkdc^{scid}II2rg^{tm1/Wj1}/Szj (NSG) mice to assess in vivo anti-tumor efficacy of VH chimeric antigen receptors (VCARs). Untreated mice (PBS) served as controls. For the in vivo studies, all CAR-T cells were produced using PiggyBac (PB) delivery of the VCAR plasmids. The mice were injected in the axilla with MDA-MB-468 (n=17 to account for variability) and treated when tumors were established (100-200 mm3 by caliper measurement, 39 days post implantation). Mice (n=4/group and staged by tumor volume) were treated with a ‘stress’ dose (2.5×10\hat{ }6) of CAR-Ts by IV injection. Whole blood was collected every 7 days and tumor volume was assessed by caliper measurement every 3 days until study completion at day 32 post CAR-T infusion.

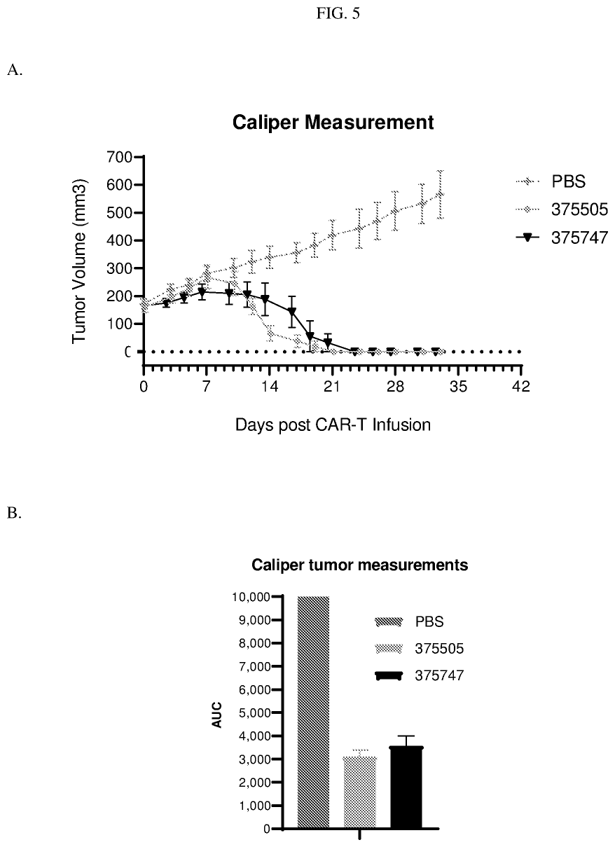

FIG. 5, Panel A, is a graph showing tumor progression as a function of days post CAR-T infusion for the indicated antibody constructs in the pre-clinical evaluation. Tumor volume assessment was completed by caliper measurement. CAR-T cells comprising the indicated antibody constructs (Clone ID: 375505 and 375747) showed strong tumor control compared to untreated mice. For example, shortly after treatment, a sharp decline in tumor volume is seen for Clone ID Nos 375505 and 375747. In contrast, tumor volume continued to climb in untreated mice after treatment administration of the control.

FIG. 5, Panel B, is a bar chart showing the area under curve (AUC) of the graph shown in FIG. 5, Panel A, for the indicated antibody constructs. Untreated mice showed substantially higher AUC in comparison to Clone ID Nos 375505 and 375747.

### Example 6

### T-Cell Expansion for CAR-T Assessment in vivo

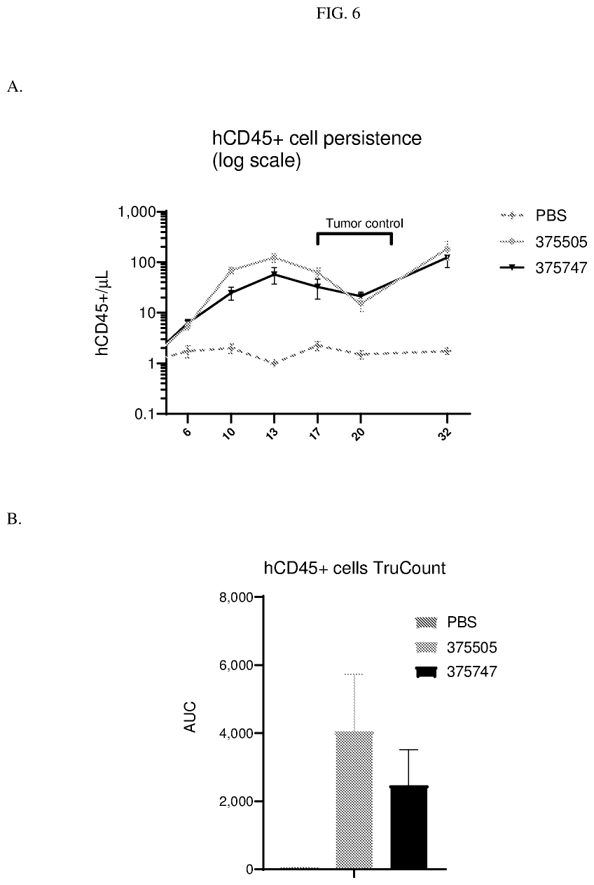

The same pre-clinical evaluation described in Example 5 was used to evaluate T-cell expansion. FIG. 6, Panel A, is a graph showing in vivo T-cell count in blood as a function of days post CAR-T infusion for the indicated antibody constructs. Robust T-cell expansion for mice treated with CAR-T cells comprising the indicated antibody constructs (Clone ID: 375505 and 375747) is shown. On day 0 the mice showed a low T-cell count. Following treatment with CAR-T cells comprising the indicated antibody constructs (Clone ID: 375505 and 375747), a sharp increase in T-cell count was seen compared with no change in untreated mice. For untreated mice, the T-cell count remained consistently low and unchanged over the course of the study.

FIG. 6, Panel B, is a bar chart showing the area under curve (T-cell count totals) of the graph shown in FIG. 6, Panel A, for the indicated antibody constructs. The total T-cell counts for both antibody constructs are significant compared to those for untreated mice.

### Example 7

### Tumor Control by CAR-T-Mediated T-Cell Activation Assessment in vivo

A pre-clinical evaluation of tumor growth was assessed for antibody constructs corresponding to Clone ID Nos. 375505 and 375747 using a murine xenograft model of ovarian cancer. A GFP- and luciferase- expressing adenocarcinoma OVCAR-3 cell line (OVCAR-3.lucGFP; 5×10\hat{ }6 cells) was injected subcutaneously into NOD.Cg-Prkdc^{scid}II2rg^{tm1Wj1}/Szj (NSG) mice to assess in vivo anti-tumor efficacy of VH chimeric antigen receptors (VCARs). Untreated mice (PBS) served as controls. For the in vivo studies, all CAR-T cells were produced using PiggyBac (PB) delivery of the VCAR plasmids. The mice were injected with tumor cells (n=17 to account for variability) and treated when tumors were established (flux greater than 1×10\hat{ }8 photons/sec by bioluminescent imaging (BLI), 14 days post implantation). Mice (n=4/group and staged by tumor volume) were treated with a ‘stress’ dose (8×10\hat{ }6) of CAR-Ts by IV injection. Whole blood was collected every 7 days and tumor volume was assessed by BLI measurement every 7 days until study completion at day 56 post CAR-T infusion.

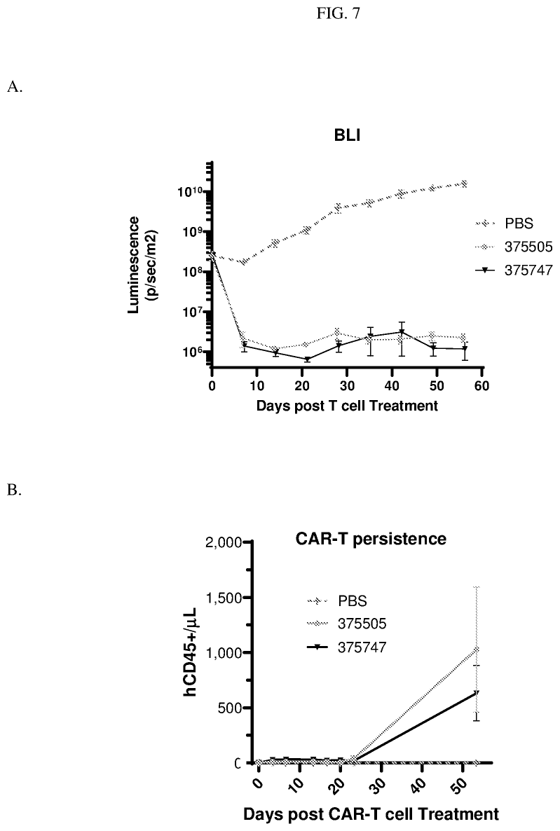

FIG. 7, Panel A, is a graph showing tumor progression as a function of days post CAR-T infusion for the indicated antibody constructs in the pre-clinical evaluation. Tumor volume assessment was completed by BLI measurement. CAR-T cells comprising the indicated antibody constructs (Clone ID: 375505 and 375747) showed strong tumor control compared to untreated mice. For example, shortly after treatment, a sharp decline in tumor volume is seen for Clone ID Nos 375505 and 375747. In contrast, tumor volume continued to climb in untreated mice after treatment administration of the control.

FIG. 7, Panel B, is a line graph showing T-cell persistence in blood measured over time for the indicated antibody constructs. Untreated mice showed substantially higher AUC in comparison to Clone ID Nos 375505 and 375747.

### Example 8

### T-Cell Expansion for CAR-T Assessment in vivo

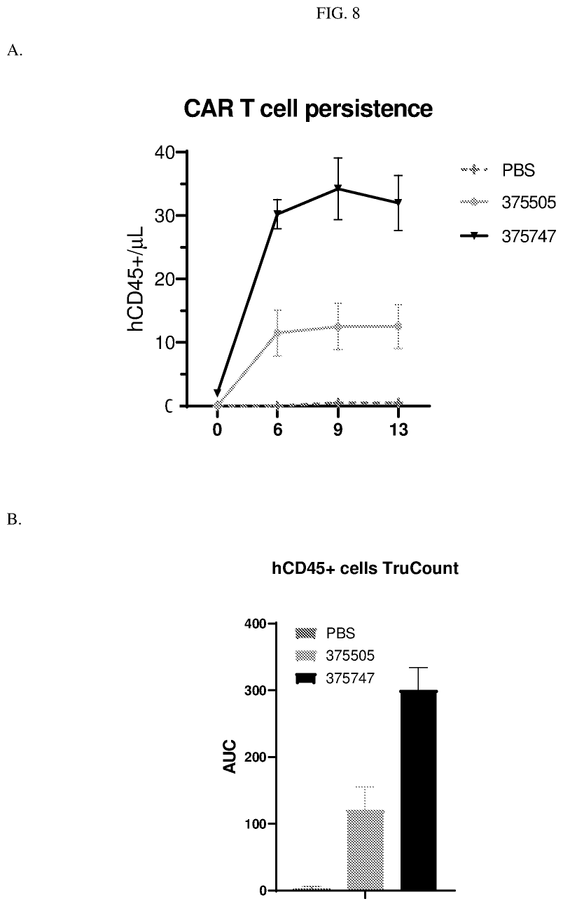

The same pre-clinical evaluation described in Example 7 was used to evaluate T-cell expansion. FIG. 8, Panel A, is a graph showing in vivo T-cell count in blood as a function of days post CAR-T infusion for the indicated antibody constructs. Robust T-cell expansion for mice treated with CAR-T cells comprising the indicated antibody constructs (Clone ID: 375505 and 375747) is shown. On day 0 the mice showed a low T-cell count. Following treatment with CAR-T cells comprising the indicated antibody constructs (Clone ID: 375505 and 375747), a sharp increase in T-cell count was seen compared with no change in untreated mice. For untreated mice, the T-cell count remained consistently low and unchanged over the course of the study.

FIG. 8, Panel B, is a bar chart showing the area under curve (T-cell count totals) of the graph shown in FIG. 8, Panel A, for the indicated antibody constructs. The total T-cell counts for both antibody constructs is significant compared to those for untreated mice.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An antibody that binds to MUC1-C, comprising a heavy chain variable region comprising:
(a) a CDR1 sequence comprising two or fewer substitutions in any one of the amino acid sequences of SEQ ID NOs: 1 or 4; and/or
(b) a CDR2 sequence comprising two or fewer substitutions in any one of the amino acid sequences of SEQ ID NOs: 2 or 5; and/or
(c) a CDR3 sequence comprising two or fewer substitutions in any one of the amino acid sequences of SEQ ID NOs: 3 or 6.

2. The antibody of claim 1, wherein said CDR1, CDR2, and CDR3 sequences are present in a human framework.

3. The antibody of claim 1, further comprising a heavy chain constant region sequence in the absence of a CH1 sequence.

4. The antibody of any one of claims 1-3, comprising:
(a) a CDR1 sequence selected from the group consisting of SEQ ID NOs: 1 and 4; and/or
(b) a CDR2 sequence selected from the group consisting of SEQ ID NOs: 2 and 5; and/or
(c) a CDR3 sequence selected from the group consisting of SEQ ID NOs: 3 and 6.

5. The antibody of claim 4, comprising:
(a) a CDR1 sequence selected from the group consisting of SEQ ID NOs: 1 and 4; and
(b) a CDR2 sequence selected from the group consisting of SEQ ID NOs: 2 and 5; and
(c) a CDR3 sequence selected from the group consisting of SEQ ID NOs: 3 and 6.

6. The antibody of claim 5, comprising:
(a) a CDR1 sequence of SEQ ID NO: 1, a CDR2 sequence of SEQ ID NO: 2, and a CDR3 sequence of SEQ ID NO: 3; or
(b) a CDR1 sequence of SEQ ID NO: 4, a CDR2 sequence of SEQ ID NO: 5, and a CDR3 sequence of SEQ ID NO: 6.

7. The antibody of any one of claims 1-3, comprising a heavy chain variable region having at least 95% sequence identity to any of the sequences of SEQ ID NOs: 7-8.

8. The antibody of claim 7, comprising a heavy chain variable region sequence selected from the group consisting of SEQ ID NOs: 7-8.

9. The antibody of claim 8, comprising a heavy chain variable region sequence of SEQ ID NO: 7.

10. The antibody of claim 8, comprising a heavy chain variable region sequence of SEQ ID NO: 8.

11. An antibody that binds to MUC1-C, comprising a heavy chain variable region comprising CDR1, CDR2 and CDR3 sequences in a human VH framework, wherein the CDR sequences are sequences having two or fewer substitutions in a CDR sequence selected from the group consisting of SEQ ID NOs: 1-6.

12. The antibody of claim 11, comprising a heavy chain variable region comprising CDR1, CDR2 and CDR3 sequences in a human VH framework, wherein the CDR sequences are selected from the group consisting of SEQ ID NOs: 1-6.

13. An antibody that binds to MUC1-C, comprising a heavy chain variable region comprising:
(a) a CDR1 sequence of SEQ ID NO: 1, a CDR2 sequence of SEQ ID NO: 2, and a CDR3 sequence of SEQ ID NO: 3, in a human VH framework; or
(b) a CDR1 sequence of SEQ ID NO: 4, a CDR2 sequence of SEQ ID NO: 5, and a CDR3 sequence of SEQ ID NO: 6, in a human VH framework.

14. The antibody of any one of claims 1 to 13, which is in a CAR-T format.

15. The antibody of any one of claims 1-13, which is multi-specific.

16. The antibody of claim 15, which is bispecific.

17. The antibody of claim 16, which binds to two different MUC1-C proteins.

18. The antibody of claim 16, which binds to two different epitopes on the same MUC1-C protein.

19. The antibody of claim 15, which binds to an effector cell.

20. The antibody of claim 15, which binds to a T-cell antigen.

21. The antibody of claim 20, which binds to CD3.

22. The antibody of claim 21, comprising:
(a) a heavy chain variable region comprising:
(i) a CDR1 sequence of SEQ ID NO: 9, a CDR2 sequence of SEQ ID NO: 10, and a CDR3 sequence of SEQ ID NO: 11, in a human VH framework; or
(ii) a CDR1 sequence of SEQ ID NO: 12, a CDR2 sequence of SEQ ID NO: 13, and a CDR3 sequence of SEQ ID NO: 14, in a human VH framework; and
(b) a light chain variable region comprising a CDR1 sequence of SEQ ID NO: 15, a CDR2 sequence of SEQ ID NO: 16, and a CDR3 sequence of SEQ ID NO: 17, in a human VL framework.

23. The antibody of claim 22, comprising:
(a) a heavy chain variable region comprising:
(i) a heavy chain variable region sequence having at least 95% sequence identity to SEQ ID NO: 18; or
(ii) a heavy chain variable region sequence having at least 95% sequence identity to SEQ ID NO: 19; and
(b) a light chain variable region sequence having at least 95% sequence identity to SEQ ID NO: 20.

24. The antibody of claim 23, comprising:
(a) a heavy chain variable region comprising:
(i) a heavy chain variable region sequence comprising SEQ ID NO: 18; or
(ii) a heavy chain variable region sequence comprising SEQ ID NO: 19; and
(b) a light chain variable region sequence comprising SEQ ID NO: 20.

25. A bispecific three-chain antibody-like molecule (TCA) that binds to MUC1-C and CD3, comprising:
(a) a first polypeptide consisting of SEQ ID NO: 32;
(b) a second polypeptide selected from the group consisting of: SEQ ID NO: 33 and SEQ ID NO: 42; and
(c) a third polypeptide selected from the group consisting of: SEQ ID NO: 34, SEQ ID NO: 35, SEQ ID NO: 36, SEQ ID NO: 37, SEQ ID NO: 38, and SEQ ID NO: 39.

26. A CAR-T cell comprising a CAR comprising an extracellular antigen-binding domain that binds to MUC1-C, comprising a heavy chain variable region comprising:
(a) a CDR1 sequence of SEQ ID NO: 1, a CDR2 sequence of SEQ ID NO: 2, and a CDR3 sequence of SEQ ID NO: 3; or
(b) a CDR1 sequence of SEQ ID NO: 4, a CDR2 sequence of SEQ ID NO: 5, and a CDR3 sequence of SEQ ID NO: 6.

27. The CAR-T cell of claim 26, wherein the extracellular antigen-binding domain that binds to MUC1-C comprises a heavy chain variable region having at least 95% sequence identity to any of the sequences of SEQ ID NOs: 7-8.

28. The CAR-T cell of claim 27, wherein the extracellular antigen-binding domain that binds to MUC1-C comprises a heavy chain variable region sequence selected from the group consisting of SEQ ID NOs: 7-8.

29. The CAR-T cell of claim 28, wherein the extracellular antigen-binding domain that binds to MUC1-C comprises a heavy chain variable region sequence of SEQ ID NO: 7.

30. The CAR-T cell of claim 28, wherein the extracellular antigen-binding domain that binds to MUC1-C comprises a heavy chain variable region sequence of SEQ ID NO: 8.

31. A pharmaceutical composition comprising an antibody of any one of claims 1-25, or a CAR-T cell of any one of claims 26-30.

32. A method for the treatment of a disorder characterized by expression of MUC1-C, comprising administering to a subject with said disorder an antibody of any one of claims 1-25, a CAR-T cell of any one of claims 26-30, or a pharmaceutical composition of claim 31.

33. The method of claim 32, wherein the disorder is a cancer.

34. The method of claim 33, wherein the cancer is a carcinoma.

35. The method of claim 34, wherein the carcinoma is an adenocarcinoma or a squamous cell carcinoma.

36. The method of claim 34, wherein the carcinoma is selected from the group consisting of:
breast, non-small cell lung (NSCL), small cell lung (SSC), mesothelioma, renal cell, colorectal, ovarian, head and neck squamous cell, nasopharyngeal, gastric, prostatic, pancreatic, esophageal, and cervical carcinoma.

37. The method of claim 33, wherein the cancer is a hematological cancer.

38. The method of claim 37, wherein the hematological cancer is a myeloma.

39. The method of claim 38, wherein the myeloma is multiple myeloma (MM).

40. The method of claim 37, wherein the hematological cancer is a leukemia.

41. The method of claim 40, wherein the leukemia is chronic myeloid leukemia (CML).

42. The method of claim 37, wherein the hematological cancer is a lymphoma.

43. A polynucleotide encoding an antibody of any one of claims 1-25, or a CAR of a CAR-T cell of any one of claims 26-30.

44. A vector comprising the polynucleotide of claim 43.

45. A cell comprising the vector of claim 44.

46. A method of producing an antibody of any one of claims 1-25, the method comprising growing a cell according to claim 43 under conditions permissive for expression of the antibody, and isolating the antibody from the cell and/or a cell culture medium in which the cell is grown.

47. A method of making an antibody of any one of claims 1-25, the method comprising immunizing a UniRat animal with MUC1-C and identifying MUC1-C-binding heavy chain sequences.

48. A method of treatment, comprising administering to an individual in need an effective dose of an antibody of any one of claims 1-25, a CAR-T cell of any one of claims 26-30, or a pharmaceutical composition of claim 31.

49. Use of an antibody of any one of claims 1-25 or a CAR-T cell of any one of claims 26-30 in the preparation of a medicament for the treatment of a disease or disorder in an individual in need.

50. The antibody of any one of claims 1-25, the CAR-T cell of any one of claims 26-30, or the pharmaceutical composition of claim 31, for use in therapy in an individual in need.

51. A kit for treating a disease or disorder in an individual in need, comprising an antibody of any one of claims 1-25, a CAR-T cell of any one of claims 26-30, or a pharmaceutical composition of claim 31, and instructions for use.

52. The kit of claim 51, further comprising at least one additional reagent.

53. The kit of claim 52, wherein the at least one additional reagent comprises a chemotherapeutic drug.


SEQUENCE LISTING (43 sequences)

SEQ ID NO: 1 - protein, 8 aa, Artificial Sequence
  Description of Artificial Sequence Synthetic peptide
GFAFSGNS

SEQ ID NO: 2 - protein, 8 aa, Artificial Sequence
  Description of Artificial Sequence Synthetic peptide
ITSSGRSI

SEQ ID NO: 3 - protein, 12 aa, Artificial Sequence
  Description of Artificial Sequence Synthetic peptide
ATGGTGTSLFDY

SEQ ID NO: 4 - protein, 8 aa, Artificial Sequence
  Description of Artificial Sequence Synthetic peptide
GFTFSSHS

SEQ ID NO: 5 - protein, 8 aa, Artificial Sequence
  Description of Artificial Sequence Synthetic peptide
ISSSSNIK

SEQ ID NO: 6 - protein, 12 aa, Artificial Sequence
  Description of Artificial Sequence Synthetic peptide
ATGGTGITVLDY

SEQ ID NO: 7 - protein, 119 aa, Artificial Sequence
  Description of Artificial Sequence Synthetic polypeptide
EVQLVESGGGLVQPGGSLRLSCTASGFAFSGNSMNWVRQAPGKGLEWVAFITSSGRSIKYADSVKGRFTISRDNAKNSLYLQMNTLRDEDTALYYCATGGTGTSLFDYRGQGTLVTVSS

SEQ ID NO: 8 - protein, 119 aa, Artificial Sequence
  Description of Artificial Sequence Synthetic polypeptide
EVQLVESGGGLVQPGGSLRLSCAASGFTFSSHSMNWVRQAPGKGLEWVSFISSSSNIKKYADSVKGRFTISRDNAKNSLFLQMNSLRDEDTAVYYCATGGTGITVLDYRGQGTLVTVSS

SEQ ID NO: 9 - protein, 8 aa, Artificial Sequence
  Description of Artificial Sequence Synthetic peptide
GFTFDDYA

SEQ ID NO: 10 - protein, 8 aa, Artificial Sequence
  Description of Artificial Sequence Synthetic peptide
ISWNSGSI

SEQ ID NO: 11 - protein, 16 aa, Artificial Sequence
  Description of Artificial Sequence Synthetic peptide
AKDSRGYGDYRLGGAY

SEQ ID NO: 12 - protein, 8 aa, Artificial Sequence
  Description of Artificial Sequence Synthetic peptide
GFTFHNYA

SEQ ID NO: 13 - protein, 8 aa, Artificial Sequence
  Description of Artificial Sequence Synthetic peptide
ISWNSGSI

SEQ ID NO: 14 - protein, 16 aa, Artificial Sequence
  Description of Artificial Sequence Synthetic peptide
AKDSRGYGDYSLGGAY

SEQ ID NO: 15 - protein, 6 aa, Artificial Sequence
  Description of Artificial Sequence Synthetic peptide
QSVSSN

SEQ ID NO: 16 - protein, 3 aa, Artificial Sequence
  Description of Artificial Sequence Synthetic peptide
GAS

SEQ ID NO: 17 - protein, 9 aa, Artificial Sequence
  Description of Artificial Sequence Synthetic peptide
QQYNNWPWT

SEQ ID NO: 18 - protein, 123 aa, Artificial Sequence
  Description of Artificial Sequence Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 19 - protein, 123 aa, Artificial Sequence
  Description of Artificial Sequence Synthetic polypeptide
EVQLVESGGGLVQPGRSLRLSCAASGFTFHNYAMHWVRQAPGKGLEWVSGISWNSGSIGYADSVKGRFTISRDNAKNSLYLQMNSLRAEDTALYYCAKDSRGYGDYSLGGAYWGQGTLVTVSS

SEQ ID NO: 20 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 21 - protein, 330 aa, Homo sapiens [sequence omitted]

SEQ ID NO: 22 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 327 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 107 aa, Artificial Sequence [sequence omitted]

SEQ ID NO: 26 - protein, 453 aa, Artificial Sequence
  Description of Artificial Sequence Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 453 aa, Artificial Sequence
  Description of Artificial Sequence Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 450 aa, Artificial Sequence
  Description of Artificial Sequence Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 450 aa, Artificial Sequence
  Description of Artificial Sequence Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 229 aa, Artificial Sequence
  Description of Artificial Sequence Synthetic polypeptide
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 31 - protein, 229 aa, Artificial Sequence
  Description of Artificial Sequence Synthetic polypeptide
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 450 aa, Artificial Sequence
  Description of Artificial Sequence Synthetic polypeptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 348 aa, Artificial Sequence
  Description of Artificial Sequence Synthetic polypeptide
EVQLVESGGGLVQPGGSLRLSCTASGFAFSGNSMNWVRQAPGKGLEWVAFITSSGRSIKYADSVKGRFTISRDNAKNSLYLQMNTLRDEDTALYYCATGGTGTSLFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 35 - protein, 477 aa, Artificial Sequence
  Description of Artificial Sequence Synthetic polypeptide
EVQLVESGGGLVQPGGSLRLSCTASGFAFSGNSMNWVRQAPGKGLEWVAFITSSGRSIKYADSVKGRFTISRDNAKNSLYLQMNTLRDEDTALYYCATGGTGTSLFDYRGQGTLVTVSSGGGGSGGGGSEVQLVESGGGLVQPGGSLRLSCTASGFAFSGNSMNWVRQAPGKGLEWVAFITSSGRSIKYADSVKGRFTISRDNAKNSLYLQMNTLRDEDTALYYCATGGTGTSLFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 36 - protein, 348 aa, Artificial Sequence
  Description of Artificial Sequence Synthetic polypeptide
EVQLVESGGGLVQPGGSLRLSCAASGFTFSSHSMNWVRQAPGKGLEWVSFISSSSNIKKYADSVKGRFTISRDNAKNSLFLQMNSLRDEDTAVYYCATGGTGITVLDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 37 - protein, 477 aa, Artificial Sequence
  Description of Artificial Sequence Synthetic polypeptide
EVQLVESGGGLVQPGGSLRLSCAASGFTFSSHSMNWVRQAPGKGLEWVSFISSSSNIKKYADSVKGRFTISRDNAKNSLFLQMNSLRDEDTAVYYCATGGTGITVLDYRGQGTLVTVSSGGGGSGGGGSEVQLVESGGGLVQPGGSLRLSCAASGFTFSSHSMNWVRQAPGKGLEWVSFISSSSNIKKYADSVKGRFTISRDNAKNSLFLQMNSLRDEDTAVYYCATGGTGITVLDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 38 - protein, 477 aa, Artificial Sequence
  Description of Artificial Sequence Synthetic polypeptide
EVQLVESGGGLVQPGGSLRLSCTASGFAFSGNSMNWVRQAPGKGLEWVAFITSSGRSIKYADSVKGRFTISRDNAKNSLYLQMNTLRDEDTALYYCATGGTGTSLFDYRGQGTLVTVSSGGGGSGGGGSEVQLVESGGGLVQPGGSLRLSCAASGFTFSSHSMNWVRQAPGKGLEWVSFISSSSNIKKYADSVKGRFTISRDNAKNSLFLQMNSLRDEDTAVYYCATGGTGITVLDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 39 - protein, 477 aa, Artificial Sequence
  Description of Artificial Sequence Synthetic polypeptide
EVQLVESGGGLVQPGGSLRLSCAASGFTFSSHSMNWVRQAPGKGLEWVSFISSSSNIKKYADSVKGRFTISRDNAKNSLFLQMNSLRDEDTAVYYCATGGTGITVLDYRGQGTLVTVSSGGGGSGGGGSEVQLVESGGGLVQPGGSLRLSCTASGFAFSGNSMNWVRQAPGKGLEWVAFITSSGRSIKYADSVKGRFTISRDNAKNSLYLQMNTLRDEDTALYYCATGGTGTSLFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 40 - protein, 5 aa, Artificial Sequence
  Description of Artificial Sequence Synthetic peptide
GGGGS

SEQ ID NO: 41 - protein, 10 aa, Artificial Sequence
  Description of Artificial Sequence Synthetic peptide
GGGGSGGGGS

SEQ ID NO: 42 - protein, 450 aa, Artificial Sequence
  Description of Artificial Sequence Synthetic polypeptide
EVQLVESGGGLVQPGRSLRLSCAASGFTFHNYAMHWVRQAPGKGLEWVSGISWNSGSIGYADSVKGRFTISRDNAKNSLYLQMNSLRAEDTALYYCAKDSRGYGDYSLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 50 aa, Artificial Sequence
  Description of Artificial Sequence Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS